(19) United States
(12) Patent Application Publication
(10) Pub. No.: US 2022/0151569 A1
(43) Pub. Date: May 19, 2022

(54) System and Methods for Indicating Pre-Sympomatic Adverse Conditions in a Human

(71) Applicant: The Government of the United States as represented by the Secretary of the Army, Fort Detrick, MD (US)

(72) Inventors: Jaques Reifman, New Market, MD (US); Sidhartha Chaudhury, Baltimore, MD (US); Chenggang Yu, Boyds, MD (US); Samantha L. Hornby, Frederick, MD (US)

(73) Assignee: The Government of the United States as represented by the Secretary of the Army, Fort Detrick, MD (US)

(21) Appl. No.: 17/525,887

(22) Filed: Nov. 13, 2021

Related U.S. Application Data
(60) Provisional application No. 63/113,711, filed on Nov. 13, 2020.
(60) Provisional application No. 63/114,490, filed on Nov. 16, 2020.
(60) Provisional application No. 63/115,553, filed on Nov. 18, 2020.
(60) Provisional application No. 63/116,014, filed on Nov. 19, 2020.

(51) Int. Cl. A61B 5/00; A61B 5/024; A61B 5/0205; A61B 5/11

(52) U.S. Cl. CPC A61B 5/7275; A61B 5/02438; A61B 5/0205; A61B 2562/0219; A61B 5/746; A61B 5/742; A61B 5/681; A61B 5/1118

(57) ABSTRACT

A method and a system analyze an individual's physiological data to identify aberrant vital-signs patterns and estimate an infection probability. The identification includes determining a score based on the current vital-sign measurements, and the mean and standard deviation of baseline vital-signs values. In one embodiment, the mean and the standard deviation are selected based on the corresponding baseline time window and activity bin of the current vital-sign measurement. The score is identified as normal when between two thresholds and aberrant outside of those two thresholds. Estimation of infection probability pₜ, at monitoring time t, is a recursive estimate of the infection probability of infection pₜ:

$${p}_{t} = \frac{1}{1 + {( \frac{{p}_{h}}{{p}_{i}} )}^{n} {( \frac{1 - {p}_{h}}{1 - {p}_{i}} )}^{k} ( \frac{1}{{p}_{t - 1}} - 1 )}$$

where pₕ and pᵢ, respectively, represent the probability of observing aberrant vital-signs in healthy and infected individuals, and n and k, respectively, represent the number of aberrant and normal scores in the monitoring time window. In one implementation, the vital-sign used is the individual's heart rate.

### I. FIELD OF THE INVENTION

In at least one embodiment, a method and a system provide an indication of a pre-symptomatic adverse condition such as an infection (or illness) in a human (or individual) being monitored. In a further embodiment, the indication is based on the level of any deviation from baseline vital-sign data taking into account the time of day and the activity level of the individual and the probabilities that healthy and infected individuals would have deviations (e.g., aberrant vital-sign measurements) as compared to normal vital-sign measurements. In at least one further embodiment, the deviation determination is based on a vital-sign score and a threshold range (or a pair of thresholds). In a further embodiment to the above, the vital-sign monitored is the individual's heart rate. In an alternative or further embodiment, multiple vital-signs are monitored.

### II. BACKGROUND

Rapid advances in sensor technology have led to the development of non-invasive wearable devices that continuously monitor an individual's vital-signs, for example as described in U.S. Pat. Nos. 8,475,368; 9,697,468; and 10,624,550 and U.S. Pat. App. Pub. No. 2014/0275854 A1. The development of wearable monitoring devices and technologies is hampered by several gaps or limitations, including, for example, the lack of: 1) available continuous vital-sign data in humans at specific times to establish baseline vital-sign data for the individual, 2) one or more algorithms that identify deviations in one or more vital-signs at comparative times from baseline vital-sign data for the individual, and 3) an appropriate clinical human challenge model that can accurately validate the system and methods.

Early and accurate pre-symptomatic detection of adverse physical events and pathogenic infections in humans is critical for rapid deployment of countermeasure strategies to mitigate exposure, including medical intervention, subject isolation, or identification of sources of exposure that are essential to maintaining civilian and military health protection. Early detection also has the potential to reduce the spread of infectious diseases, as well as early diagnosis and intervention, leading to improved treatment outcomes.

Spurred by the COVID-19 pandemic, there has been a renewed focus on the possible use of wearable devices to detect infection, with some studies enrolling thousands of subjects to voluntarily provide wearable data collected from commercially available devices, such as the Fitbit (Fitbit, Inc., San Francisco, Calif.), along with symptom surveys and testing results. While these observational studies are vital for demonstrating the potential large-scale use of wearable devices for infection detection, they have several limitations. First, these studies are based on self-reported symptoms and infection cases, and testing is typically only sought after individuals are symptomatic. While such studies can be useful to determine time-to-detection prior to symptom onset, they do not allow a determination of the time-to-detection from exposure because the time of exposure is not known. Second, detection sensitivity in these studies is biased towards symptomatic cases of infection because asymptomatic cases would typically be untested and thus unreported. Third, assessment of detection specificity in these studies is difficult because of the lack of knowledge of the non-reporting rate—negative subjects who had symptoms but did not report them or were tested but not counted. In contrast to these observational studies, controlled human-infection studies, in which human subjects are deliberately infected with a pathogen in a controlled clinical environment and then closely monitored for symptoms and infection, provide an essential complementary approach for developing and evaluating infection-detection technologies.

There have been a few observational studies using commercially available smartwatch-based wearable devices to detect infection during the COVID-19 pandemic, most finding that infection is associated with abnormal resting heart rate (Mishra, Quer, and Natarajan), respiration rate (Natarajan and Miller), and sleep and activity (Mishra and Quer). A separate study using a ring-based wearable device reported abnormal heart rate and skin temperature readings in infected subjects (Smarr). Because time of exposure in these studies is not known, early detection is difficult to evaluate. Nonetheless, two studies report observing physiological changes prior to the onset of symptoms in some percentage of infected subjects. Comparison of these studies with research by the inventors is difficult because of differences in methodology and disease, but the sensitivity and specificity observed in these studies are comparable to what we report here: 50-70% sensitivity at 80% specificity, with pre-symptomatic or early detection in 60% of cases in retrospective analysis. Although different infectious diseases have different time courses and severities, the physiological data collected in all of these studies—heart rate, skin temperature, and activity—are highly generalizable and likely non-specific to any particular disease.

Many of these studies and other works on infection detection have resulted in proposals to use resting heart rate comparisons, which restricts and limits the time periods during the course of the day that may be used and may lead to inaccurate results from the small sampling sizes imposed by such restriction. The proposals are further limited and impacted by not taking into account that an individual's heart rate and other vital signs vary during the course of the day because of circadian rhythms (e.g., about 10-20 beats per minute for heart rate), which affects the comparison of resting heart rates taken at different times of the day. An individual's heart rate and vital signs can also be impacted by extraneous factors such as stress, environmental temperature, and ambient light.

The COVID-19 pandemic has had an immense effect on both civilian and military operations, negatively impacting the economy and force health protection and readiness. For example, it has adversely impacted the overall economy and both the fitness of health professionals and military troops to perform their missions and their capability to train. There are additional industries that do not have the ability to transition to a remote work model and still be active such as the industrial and retail sectors where physical distancing is impractical, for example food and meat processing. The ability to rapidly screen and isolate infected individuals early, before they can infect others, is vital for reducing the spread of a virus and/or minimizing impact on the economy and restoring force protection and readiness.

### III. SUMMARY

The different embodiments discussed in this disclosure have a common theme that the method and system take into account circadian rhythm and activity level to estimate a probability of infection for an individual. In several embodiments, the model is customizable for individualized real-time prediction after establishment of a baseline for the individual. In at least one embodiment, the model is device agnostic and could be implemented in conjunction with any data source that provides heart rate (or the vital sign(s) being used) and activity data for the individual to enable detection of an adverse condition based on abnormalities in heart rate (or other monitored vital sign(s)) patterns. One advantage to using an activity-level specific, percentile-based approach to detecting abnormal readings (heart rate or other vital sign readings) is that it accounts for the greater noise in the measured data to baseline data collected at the same activity level. Thus, as long as the noise with respect to activity level is comparable between the baseline and detection (or monitoring) periods, increased noise at higher activity levels will not result in higher abnormal frequencies.

A wearable device feasibility study was conducted using the commercially available Samsung Gear S3 smartwatch to monitor heart rate, skin temperature, and body acceleration data in subjects as they underwent a controlled human malaria infection (CHMI). As part of the study, a model with particular parameters was developed that analyzes heart rate and activity data, identifies aberrant heart-rate patterns, and estimates in real time a probability of infection. The findings showed that this model implementation was able to detect infection prior to the clinical-grade blood-based diagnostic with a sensitivity of 67%, with a false positive rate of 6% per week. While this false-positive rate may be too high for general population use where it might greatly exceed the prevalence rate of infection, in circumstances with the potential for high prevalence or susceptibility this could serve as a vital screening tool for detecting and treating infections. Such examples of high disease prevalence (>10%) include diarrhea or dysentery among international travelers, malaria during the rainy season in a country where it is endemic, influenza and adenovirus infections in basic training for military personnel, and even health-care settings during an outbreak or pandemic response. For society, this additional screening could impact future responses to outbreaks and pandemics in terms of the use of restrictions. With adjustment of the model parameters, the sensitivity and false positive rate can be adjusted.

The study underlying the discussed models is unique among wearable infection studies in several key aspects: it used a controlled infection model in order to precisely establish the time of exposure and onset of infection; it used a Bayesian-based model that accounts for circadian rhythm and activity level to estimate an individualized probability of infection and enable real-time inferences; and the developed model is device-agnostic and disease non-specific, allowing its integration with any wearable device that accurately measures heart rate and activity for any infection event that generates abnormalities in heart rate patterns. These findings underscore the value of using controlled infection studies to develop early infection-detection technologies, demonstrate the feasibility of using wearable devices to detect infection, and highlight the potential use of this technology in screening efforts.

In at least one embodiment, the method analyzes the wearable physiological data in two stages: 1) identification of aberrant heart-rate patterns and 2) estimation of infection probability. Classification of aberrant heart rates: from the raw heart rate (HR), a normalized heart rate score S was computed, with S=(HR−μ)/σ, where μ and σ denote mean and standard deviation, respectively, of raw baseline heart rates. In at least one embodiment, the mean and the standard deviation are selected based on the corresponding time window and activity bin for the raw heart rate data. Then two thresholds (upper and lower) are set and to classify the heart rate scores between the two thresholds as normal and those outside of the two thresholds as aberrant. For each individual, the two thresholds may be determined by allowing a fixed percentage of aberrant heart rates, for example, by no more than 5%, in the baseline period. Estimation of infection probability pₜ: Bayesian statistics were employed to accumulate evidence of aberrant heart rate patterns over time and assess the likelihood of infection, based on the accumulated evidence. In particular, at time t, a recursive estimate of the probability of infection pₜ is:

$${p}_{t} = \frac{1}{1 + {( \frac{{p}_{h}}{{p}_{i}} )}^{n} {( \frac{1 - {p}_{h}}{1 - {p}_{i}} )}^{k} ( \frac{1}{{p}_{t - 1}} - 1 )}$$

where pₕ denotes the probability of observing aberrant heart rates in healthy subjects, pᵢ represents the probability of observing aberrant heart rates in infected subjects, and n and k represent, respectively, the number of aberrant and normal heart rate measurements (or scores) in a time window between time t−1 and time t. These represent newly collected evidence since the previous estimation of infection probability pₜ₋₁ at time t−1. The preliminary results have demonstrated the ability of the Bayesian algorithm to correctly predict infection during the incubation period, as early as 3 to 4 days prior to the onset of symptoms and parasitemia diagnosis with an FDA-approved test (see, e.g., FIGS. 10A-10C).

According to at least one embodiment, it is possible to determine whether a person has a pre-symptomatic adverse condition such as an infection based on differences between current vital-sign data (e.g., heart rate) and baseline vital-sign data where the vital-sign data is categorized based on the time of day and the activity level. Each day is divided into time windows with each time window having multiple activity levels to facilitate the comparison between current vital-sign data and baseline vital-sign data since the current (or monitoring) and baseline time window and activity level combinations (or bins) can be matched. The difference between the current vital-sign data and the baseline vital-sign data is measured by a normalized score compared to a threshold to determine if the vital-sign is normal or aberrant, which is used in determining an infection probability along with the previous infection probability and the probability an aberrant vital sign would occur for healthy and infected individuals. In at least one embodiment, the infection probability is provided to a receiving individual, for example, the monitored individual, a supervisor, a medical provider, and/or another individual or entity. In at least one embodiment to any of the monitoring embodiments in the remainder of this summary, the probability allows for the receiving individual to take action, for example, obtaining a medical diagnostic test or otherwise having a medical examination of the monitored individual for an adverse condition, such as an infection, and/or isolating the monitored individual.

In at least one embodiment, there is a method for determining a probability of a pre-symptomatic adverse condition for an individual using a heart rate of the individual, the method including: collecting baseline heart rate data and activity information for the individual over multiple days; separating the heart rate data into heart rates for time windows into bins based on a time of day and an activity level derived from the activity information; determining a baseline mean and a baseline standard deviation for the heart rates for each time window and activity bin and at least one threshold based on all of the baseline heart rate data and activity information; after the baseline mean, the baseline standard deviation, and the threshold(s) are determined, collecting (or monitoring) ongoing heart rate data and activity information for the individual, selecting a time window and activity bin based on activity information for that monitoring time window, calculating a heart rate score based on the heart rate data for the monitoring time window and the baseline mean and the baseline standard deviation for that baseline time window and activity bin corresponding to the monitoring time window and activity bin, classifying the heart rate score as normal or aberrant based on how the heart rate score compares with the threshold(s), calculating a probability using the classification as normal/aberrant, aberrant heart rate probabilities for a healthy person and an infected person, and after the first probability calculation, the previous probability, providing (or displaying or transmitting) the probability to the individual and/or another individual, and repeating the ongoing monitoring steps.

In at least one embodiment, there is a method for determining a probability of a pre-symptomatic adverse condition for an individual, the method including: collecting baseline vital-sign data and activity information for the individual over multiple days; separating the vital-sign data into vital-sign measurements for time window and activity bins based on the time of day and activity level derived from the activity information; determining a baseline mean and a baseline standard deviation for the vital-sign measurements for each time window and activity bin and at least one threshold based on all of the baseline vital-sign data and activity information; after the baseline mean, the baseline standard deviation, and the threshold(s) are determined, collecting ongoing vital-sign data and activity information for the individual, selecting a monitoring time window and activity bin based on activity information for that monitoring time window, calculating a vital-sign score based on the vital-sign data for the monitoring time window and the baseline mean and the baseline standard deviation for the time window and activity bin corresponding to the monitoring time window and the activity bin, classifying the vital-sign score as normal or aberrant based on how the vital-sign score compares with the threshold(s), calculating a probability using the classification as normal/aberrant, an aberrant vital-sign probability for a healthy person, an aberrant vital-sign probability for an infected person, and after the first probability calculation, the previous probability, providing (or displaying or transmitting) the probability to the individual and/or another individual, and repeating the monitoring steps.

In at least one embodiment, there is a method for establishing vital-sign baseline characteristics for an individual where the baseline characteristics include a mean, a standard deviation, and at least one threshold in which to compare future vital-sign measurements taken after the baseline characteristics are established, the method including: receiving baseline vital-sign data and activity information from at least one body worn measurement device for the individual over at least 10 days, at least 14 days, or at least 21 days; separating the vital-sign data into vital-sign measurements for different time window and activity bins based on the time of day and the activity level derived from the activity information; determining a baseline mean and a baseline standard deviation for the vital-sign measurements for each time window and activity bin; for each time window with baseline heart rate data and activity information, calculating a vital-sign score based on the vital-sign measurement for that time window, the baseline mean and the baseline standard deviation for the time of day and activity bin combination corresponding to the time window and activity information; and determining at least one threshold based on the vital-sign scores.

In at least one embodiment, there is a method for determining a probability of a pre-symptomatic adverse condition for an individual using a baseline mean, a baseline standard deviation and a threshold for a vital-sign in the individual being monitored, the method including: collecting vital-sign data and activity information for the individual, selecting a time window and activity bin based on activity information for that monitoring time window to which the corresponding collected vital-sign data will be associated, calculating a vital-sign score based on the vital-sign data for the monitoring time window and the baseline mean and the baseline standard deviation for that baseline time window and activity bin corresponding to the monitoring time window and activity bin, classifying the vital-sign score as normal or aberrant based on how the vital-sign score compares with the threshold(s), calculating a probability using the classification as normal/aberrant, aberrant vital-sign probabilities for a healthy person and an infected person, and after the first probability calculation, the previous probability, providing the probability to the individual and/or another individual to alert at least one of the individuals of the likelihood of pre-symptomatic adverse condition when the probability exceeds a probability threshold allowing for action to be taken; and repeating the above steps at least one time.

Further to the above method embodiments, there are a variety of further embodiments that may be used in a variety of ways and combinations as outlined in the following paragraphs. Based on this summary, one of ordinary skill in the art should appreciate that the individual's heart rate could be used as the relevant vital-sign.

Further to the above embodiments and any other method embodiment in this summary, the activity level may be derived from a data source providing metabolic equivalents (METs), activity information from a 3-axis accelerometer, data input from the individual or another, or some combination. The number of activity levels may range between 1 and 4, and more particularly 3. In at least one embodiment, the activity levels for the activity bins may be sleeping (or relaxing), office work, walking, and exercising, respectively. In a further embodiment, the activity level associated with exercising is omitted.

Further to the above embodiments and any other method embodiment in this summary, the number of baseline and/or monitoring time windows may range between 8 and 96 and/or have has a length equal to 15 minutes, 30 minutes, 45 minutes, 60 minutes, 90 minutes, or 120 minutes. Over a 24-hour day, the length for the time window may vary, for example between overnight and during the day. Alternatively or further to the embodiments of this paragraph, the time window used for vital-sign data may be an expanded time window that is longer than the time window. In a further embodiment, the expanded time window is centered about the corresponding time window.

Further to the above embodiments and any other method embodiment in this summary, a vital-sign score is calculated for each time window with baseline vital-sign data and activity information to produce a vital-sign score set to determine the at least one threshold. In a further embodiment, the threshold(s) is established by creating a distribution or a distribution curve of the vital-sign scores, selecting a lower vital-sign score and a vital-sign score from the vital-sign scores that represent a lower threshold percentage or number and a higher threshold percentage or number, respectively, calculating a lower threshold using the lower vital-sign score, calculating a higher threshold using the higher vital-sign score, and optionally setting the threshold range using the lower threshold and the higher threshold. In a further embodiment, the lower threshold percentage removes the lowest 5% of vital-sign scores and the higher threshold percentage removes the highest 5% of vital-sign scores. In an alternative embodiment, the threshold is established by creating a distribution or a distribution curve of the vital-sign scores, determining a score standard deviation and a score mean for the vital-sign scores, setting a lower threshold equal to a threshold multiple of the score standard deviation below the score baseline mean, setting a higher threshold equal to the threshold multiple of the score standard deviation above the score baseline mean, and optionally setting the threshold range using the lower threshold and the higher threshold or using the lower and higher thresholds.

In a further embodiment to the above embodiments and any other method embodiment in this summary, when the monitoring (or baseline) time window includes multiple activity levels for the individual, subdividing the vital-sign data based on the corresponding activity levels and calculating the vital-sign score for each subdivision. For the monitoring phase, using the score to determine whether each subdivision for that time window is aberrant or normal. The total number of aberrant or normal determinations for one time window is between 1 and the number of activity levels.

In a further embodiment to the above embodiments and any other method embodiment in this summary, the vital-sign data is averaged over 5 second intervals, optionally non-overlapping intervals, to provide up to 360 data points per thirty-minute time window. In an alternative embodiment, a moving average over a 15-minute window is used for each data point.

In a further embodiment to the above embodiments and any other method embodiment in this summary, alerting the individual when the probability exceeds a probability threshold to allow the individual to seek medical assistance (e.g., a medical diagnostic test or medical examination) or take other action. In a further embodiment, wherein the probability threshold is set at a level appropriate for infection frequency in an area in which the person is present such that the probability threshold is the lower of 50% or 100% minus a current infection rate.

In a further embodiment to the above embodiments and any other method embodiment in this summary, the vital-sign score S is

$$S ( {t}_{i} , {a}_{j} ) = \frac{V S ( {t}_{i} , {a}_{j} ) - m ( {t}_{i} , {a}_{j} )}{S D ( {t}_{i} , {a}_{j} )}$$

where tᵢ is the time window i in a day, aⱼ is the activity level j, VS is a mean of the vital-sign measurements based on vital-sign data for that time window (or optionally the expanded time window) and for that activity level, m is the baseline mean and SD is the baseline standard deviation. In at least one embodiment, there is a bin for each combination of time window and activity level, and in a further embodiment the activity levels exclude higher activity levels, for example from strenuous exercising.

In a further embodiment to the above embodiments and any other method embodiment in this summary, the probability is

$${p}_{t} = \frac{1}{1 + {( \frac{{p}_{h}}{{p}_{i}} )}^{n} {( \frac{1 - {p}_{h}}{1 - {p}_{i}} )}^{k} ( \frac{1}{{p}_{t - 1}} - 1 )}$$

where pₕ is a probability to observe an aberrant vital-sign in a healthy subject, pᵢ is a probability to observe an aberrant vital-sign in an infected subject, t is the vital-sign monitoring time window, and n and k, respectively, are the number of aberrant and normal vital-signs observed in the monitoring time window. In a further embodiment, the prior probability is multiplied by a multiplier resulting in past aberrant scores impacting the current probability less over time. In a further embodiment to the previous two embodiments, pₕ and pᵢ are predetermined to have their ratio as a parameter to optimize the probability. In a further embodiment to the previous three embodiments, the first probability calculation uses a predetermined value for the prior probability, which in a further embodiment is less than 0.2.

In a further embodiment to the above embodiments and any other method embodiment in this summary, when the baseline mean and the baseline standard deviation are not available for the particular time window and activity bin, using at least one of a prior time window and activity bin and a later time window and activity bin or alternatively a bin (or an average of bins) from a time window(s) within the neighboring 3 time windows.

In a further embodiment to the above vital-signs embodiments, the vital-sign is heart rate, pulse rate, blood pressure, blood oxygen saturation, or combinations thereof and optionally estimating a vital-sign from one or more of these vital-signs.

In a further embodiment to the above embodiments, the pre-symptomatic adverse condition is a malaria infection or a SARS-CoV-2 infection.

In at least one embodiment, there is a system including: a body worn vital-sign measurement device; and optionally a computing device in wireless communication with the measurement device, and where the measurement device and/or the computing device are configured to perform any of the above method embodiments. In at least one further embodiment, the measurement device also detects activity and/or the measurement device and/or computing device is able to receive input from the individual or another device (e.g., a cycling computer).

In at least one embodiment, there is a system for informing an individual of a probability of a pre-symptomatic adverse condition, the system including: at least one wearable device having at least one vital-sign monitor and/or an activity module; a computing device in wireless communication with the at least one wearable device, the computing device having a display and a processor configured to perform any of the above method embodiments.

In at least one embodiment, there is a wearable device to be worn by an individual and for informing the individual of a probability of a pre-symptomatic adverse condition, the device including: at least one vital-sign monitor; activity means for providing a representation of activity for the individual; a display; a processor configured to perform any of the above method embodiments.

The wearable device and system provide a non-invasive wearable platform for automated and accurate identification of pre-symptomatic infections, including malaria and SARS-CoV-2 cases. In at least one embodiment, a mobile, wearable device, system and method provide early and accurate detection of a pre-symptomatic condition in humans by continuously monitoring one or more preselected vital-signs in the individual and determining critical deviations in the one or more of the vital-signs in the individual.

In at least one embodiment, a system for indicating a pre-symptomatic adverse condition in a human where the system includes: at least one wireless wearable device for continuously measuring (or monitoring) one or more vital-signs at specific times in the individual to establish a baseline for the individual and provide ongoing monitoring after the baseline is established, the system further including a processor that stores the continuously measured vital-sign data from the individual and one or more models, each of the models continuously indicating deviations in the one or more vital-signs in the individual as compared to baseline vital-signs at comparative times in the individual; and one or more wireless networks, each wireless network in communication with the wearable device and/or the wireless mobile device, and wherein the pre-symptomatic adverse condition in the individual includes an adverse physical event or an infection in the individual including predicting the probability of infection based on the comparison between current vital-signs and baseline (healthy) vital-signs.

In at least one embodiment, a method for determining a pre-symptomatic adverse condition in an individual where the method includes: (a) continuously measuring (or monitoring) one or more vital-signs in the individual at specific times using at least one wireless wearable device and optionally a mobile device that individually or together with the wearable device have one or more models, each of the models continuously indicate deviations in the one or more vital-signs in the individual at comparative times, once a baseline is established for vital-sign data for the individual; (b) collecting, storing and processing the continuously measured vital-sign data for the individual using the one or more models with at least one wireless network in communication with the at least one wireless wearable device or optional mobile device; and (c) warning or providing the individual (and/or another person) of a pre-symptomatic adverse condition in the individual based on deviations in vital-sign data, as compared to the baseline for the individual, as indicated from the one or more models.

### IV. BRIEF DESCRIPTION OF THE DRAWINGS

The following drawings form part of the present specification and are included to further demonstrate certain embodiments of the present invention. The invention may be better understood by reference to one or more of these figures in combination with the detailed description of specific embodiments presented herein.

FIG. 1 illustrates a flowchart according to at least one embodiment of the invention.

FIG. 2 illustrates a flowchart according to at least one further embodiment of the invention.

FIG. 3 illustrates a system according to at least one further embodiment of the invention.

FIG. 4 illustrates how heart rate data may be normalized to the baseline data according to at least one embodiment of the invention.

FIG. 5 illustrates examples of wireless wearable devices, a wireless network, and a wireless computing device according to at least one embodiment of the invention.

FIG. 6 illustrates a schematic representation of a computer or other processor-based device.

FIG. 7 illustrates an example of a CHMI timeline schedule.

FIG. 8 illustrates an evaluation strategy that measures the accuracy of predictive algorithms based on baseline vital-sign values and both time-from-exposure, defined as the time between exposure and a positive prediction of infection, and time-to-illness, defined as the time between a positive prediction of infection and the clinical manifestation of that illness.

FIG. 9 illustrates a representative example from a single non-human primate (NHP) subject, in which the infection likelihood calculated for all three vital-signs resulted in a prediction of infection 1-2 days post-exposure and 2-3 days prior to the onset of fever.

FIGS. 10A-10C illustrate raw heart rate data, aberrant heart rate data, and infection probability data from before and after infection exposure. As illustrated, the time when parasitemia was detected (positive parasitemia) by an FDA-approved blood test and use of an alert threshold of 0.5, the model predicted infection more than 4 days before clinical diagnosis.

FIG. 11 illustrates the level of data collection during a portion of a CHMI study.

FIG. 12 illustrates a baseline determination in normalizing raw heart rate data based on level of activity in the CHMI study.

FIGS. 13A and 13B illustrate classification of aberrant heart rate scores in normalized heart rate data in the CHMI study for two different ranges of activity levels.

FIGS. 14A-18B illustrate an associate between aberrant normalized heart rate scores with the probability of infection for five of the subjects in the CHMI study.

FIGS. 19A-19D illustrate true positive rate factors in the CHMI study for four of the subjects using four different levels of sensitivity for the probability of infection.

FIGS. 20A-20E illustrate optimized probability models for the timeliness of the probability of infection for five of the subjects in the CHMI study with the observed parasitemia. FIG. 20F illustrates parameter optimization.

FIGS. 21A-21D illustrate aberrant heart rate detection by the model for a representative 7-day window. FIG. 21A illustrates raw heart-rate data, averaged in 30-second intervals. FIG. 21B illustrates raw activity data represented by the magnitude of acceleration vector averaged in 30-second intervals. FIG. 21C illustrates activity data used to stratify the heart-rate data by three activity levels (resting, low, and moderate), and the average heart rate in 30-minute non-overlapping windows was calculated for each activity level (low activity level shown). An individualized aberrant heart rate (AHR) threshold (lines), determined from baseline data, defines normal (circles) and aberrant heart rates (AHR; squares along or outside of the threshold lines). FIG. 21D illustrates normalized heart-rate data across all times of day into a HR score, for each activity level (low activity level shown), with the AHR threshold (lines), normal heart rates (circles), and AHRs (squares at or outside of the threshold lines).

FIGS. 22A-22D illustrate aberrant heart rates (AHR) and total heart rate (HR) distribution at different activity levels. AHR counts (FIGS. 22A and 22B) and total HR counts (FIGS. 22C and 22D) derived from wearable data collected for each subject at resting (white), low (gray), and moderate (black) activity levels. FIGS. 22A and 22C show data for subjects in the CHMI study while FIGS. 22B and 22D show data for the subjects in the control study.

FIG. 23 illustrates probability of infection as a function of Pᵢ adjusted for the expected probability of observing an AHR in an infected individual (Pᵢ) as a ratio of the probability of an AHR in a healthy individual (Pₕ=0.05) and assessed its effect on the maximum estimated probability of infection (Pₘₐₓ) during the incubation period (CHMI subjects, black) or total monitoring period (control subjects, gray). Based on the separation between CHMI and control subjects, a Pᵢ value of 1.7*Pₕ was selected to calculate Pₜ and an infection probability threshold of 50% for determining that a subject is “infected.”

FIGS. 24A-24C illustrate infection detection for three representative CHMI subjects. Pₜ (black line) and heart-rate (HR) score (aberrant, orange; normal, gray) over time from the pre-infection (Day −15 to Day 0) to post-challenge (Day 1 to Day 25) periods. Time points for malaria challenge (black vertical line), parasitemia as detected by blood smear (vertical line), and onset and duration of moderate and severe symptoms are shown for three CHMI subjects: subject 113 (strong detection performance, FIG. 24A), subject 98 (average detection performance, FIG. 24B), and subject 96 (weak detection performance, FIG. 24C).

FIGS. 25A-25B illustrate infection detection in subjects without infection or parasitemia. Pₜ (black line) and heart-rate (HR) score (aberrant, orange; normal, gray) over time. CHMI subject 128 (FIG. 25A) was challenged on Day 0 (black vertical line) but did not develop parasitemia. Control subject 009 (FIG. 25B) represents the median model performance in non-infected subjects.

### V. DETAILED DESCRIPTION

A. Overview

In at least one embodiment, a method is performed on a mobile wearable device (or a body-worn vital-sign measurement device) or a system where the method provides early and accurate detection of an adverse condition, including an infection in a human by continuously monitoring one or more preselected vital signs in the individual and determining critical deviations in one or more of the vital signs of the individual that are indicative of the adverse condition, including, but not limited to, a pre-symptomatic infection, an early stage infection or a symptomatic infection in the individual. Continuously monitoring is used to reflect sampling rates of the vital-sign measurement device, any data smoothing performed on the vital-sign measurements, and a recognition that wearable devise may not be collecting data while charging or, otherwise, not being worn. In at least one embodiment, the mobile wearable device, system and method provide an early and accurate detection of an infectious disease in a human and further provides analytical tools that include use of wearable devices to enable early warning of human exposure to a pathogen infection, facilitating rapid situational awareness and timely intervention. In a more particular embodiment, the vital sign is a heart rate of the individual. In at least one alternative embodiment, the invention may be used with non-human primates or more broadly with mammals.

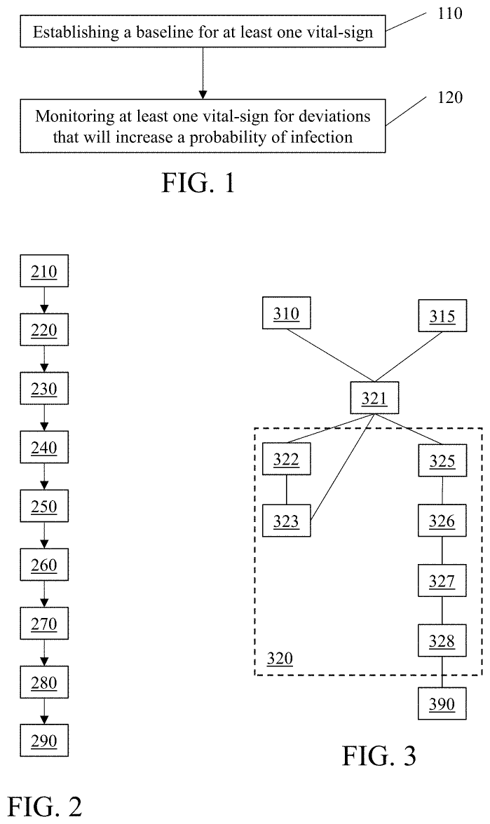

In at least one embodiment, a method for predicting the presence of an adverse condition includes establishing a baseline for each vital sign to be measured, 110, and then monitoring for a divergence in each vital sign from the established baseline that will increase the probability of an infection, 120, as illustrated in FIG. 1. In at least one embodiment, the baseline establishment and the monitoring are performed as independent methods. In a further embodiment, establishing the baseline includes collecting vital-sign data, 210, separating that vital-sign data into time window and activity bins (or “bins”) based on time windows (or time periods) and level of activity, 220, and determining characteristics for that vital-sign data, 230, as illustrated in FIG. 2. The time windows are distributed across the day (e.g., 24-hour period or calendar day) based on time of day. In a further embodiment, determining the baseline characteristics includes establishing baseline values for scoring future vital-signs and thresholds (or a threshold range) to compare the future vital-sign scores against. An individual's vital signs vary during the course of the day following a circadian rhythm (e.g., 10-20 beats per minute for heart rate) and also vary based on the level of activity an individual is doing (e.g., for many vital signs increasing depending on the metabolic load). In a further embodiment, the monitoring includes ongoing collection of vital-sign data and activity information, 240, so that the collected data can be compared against the relevant baseline vital-sign data and bin, 250, 260, to determine whether the divergence is normal or aberrant, 270, and determining a probability (or an infection score), 280, based on the number of normal and aberrant occurrences over a recent period of time and how those numbers compared to the probability that a healthy person or an infected person would have had an aberrant vital sign, and then providing the probability to the individual or another entity, 290. Examples of another entity include, but are not limited to, a supervisor or other management at the individual's workplace, a medical provider, a family member, etc. In at least one embodiment, the time windows used for the baseline phase (baseline time windows) and the monitoring phase (monitoring time windows) have the same length.

B. Baseline Phase

As illustrated in FIG. 2, establishing the baseline in at least one embodiment begins with collecting vital-sign data and activity information (collectively “raw data”) for an individual over multiple days, 210. In at least one embodiment as illustrated in FIG. 3, the vital-sign data may come from a vital-sign monitor 310, for example a smart watch, a fitness tracker, and a medical sensor (dedicated or part of another device), which may provide the vital-sign data as raw data or particular vital-sign measurements. In at least one embodiment, the activity information is obtained from an activity level module 315. In at least one embodiment, the vital-sign monitor 310 and the activity level module 315 are part of the same device. In at least one embodiment, the raw data is stored in a memory (or other storage device) 321, which in at least one embodiment is part of the vital-sign monitor 310, the activity level module 315, and/or the device with a processor 320, which may have multiple modules present on it. The length of initial vital-sign data and activity information collection to establish the baseline characteristics can be a few days to a few weeks although collection over 10-21 days, or more particularly around 14 or 21 days, which in most circumstances should provide sufficient data to establish the baseline characteristics for the individual. Other example time periods include at least 10 days, at least 14 days, and at least 21 days. In at least one embodiment, the data collection occurs over a longer period of time to increase the amount of raw data used for the baseline measurements for each time window and activity bin. In at least one embodiment, the goal is to accumulate as complete of a set of raw data for different time window and activity level (or bins). In an alternative embodiment, the raw data collection may be stopped when the time window and activity bins associated with the time the individual is awake and the lowest activity level time window and activity bins associated with the time the individual is asleep has data. In a further embodiment, the raw data is collected until a majority of the time window and activity bins have multiple vita-sign measurements.

In a further embodiment, for each baseline time window, the collected raw data over an expanded time window is used, which expanded time window is longer than the baseline time window, for example, if the baseline time window is 30 minutes long, then an additional 2.5 hours (or other time range between 30 minutes and 4 hours) might be added so that the vital-sign data across the expanded time window is aggregated together to smooth the vital-sign data out for the baseline time window. The additional time might be some split of the example 2.5 hours between prior to and after the baseline time window of interest such that the preceding time might range between 0-2.5 hours, greater than 30 minutes or one hour, or 1.25 hours and the time after might range between 0-2.5 hours, greater than 30 minutes or one hour, or 1.25 hours such that the balance is 2.5 hours. In an alternative embodiment, the additional time is 3 hours around the baseline time window. Based on this explanation, it should be apparent that different combinations and lengths of expanded and baseline time windows might be used even when the total time before and after does not equal the example 2.5 or 3 hours. In at least one embodiment, the vital-sign data is smoothed by averaging (or combining) multiple individual measurements into one vital-sign measurement (or data point). In at least one embodiment, with a sampling rate of 1 Hz, the vital-sign data is averaged in non-overlapping 5-second windows with a moving average being taken using a 15-minute window.

Further examples of baseline time window lengths include 15 minutes, 45 minutes, 60 minutes, 90 minutes, 120 minutes, 150 minutes, 180 minutes, or a length of a monitoring time window. The number of resulting baseline time windows for a particular day could vary from 8 to 72 time windows based on the length of individual baseline time windows, in a further embodiment there are 48 time windows of 30 minutes each. In an alternative embodiment, the length of time windows overnight during typical sleeping hours are longer than the length of time windows during typical hours that people are awake. The number of baseline (or monitoring) time windows is a balance of specificity and sensitivity.

Either after completion of the raw data collection or during the raw data collection, separating (or classifying) the vital-sign data as vital-sign measurements into baseline time window (based on the time of day) and activity (derived based on the level of activity) bins, 220. The separating (or classifying) may be done with hardware, software or some combination such as a processor 320 running software (i.e., a classification module 322) where the processor is located in a smart watch, a fitness tracker, a smart phone, or a computer such that when the data/information sources are on a different device there is wireless communication to transmit the data/information to the processor. In a further embodiment, the vital-sign data is further segregated within the baseline time window based on the activity level to which the vital-sign data is associated, which may lead to vital-sign data being allocated to two or more time window and activity bins for that baseline time window for that day. In a still further embodiment, there is a threshold amount of vital-sign data required for use otherwise the vital-sign data may be disregarded for the purpose of establishing the baseline time window and activity bin for that particular time period of that day. In at least one embodiment, the threshold amount is sufficient data to be statistically analyzed, for example, 5 or 10 minutes of data.

There are a variety of ways that the level of activity for the individual might be determined from actigraphy data, 2-axis or 3-axis accelerometer data, analysis of location and movement data to provide metabolic equivalents (METs) data, and/or data entered by the individual through a graphical user interface or some combination, for example based on type of activity. Each of these sources are examples of the activity level module 315, or, alternatively, activity means for providing a representation of activity for the individual. The accelerometer data is representative of the level of movement of the measuring device and as such would indicate the level of activity of the individual. In as least one embodiment, 3-axis accelerometer data is collected at 25 Hz (or higher frequency, for example, 50 Hz or 100 Hz), averaged in a non-overlapping 5-second window along each axis to be converted into a magnitude of acceleration vector, which is used to calculate a MET using an algorithm, for example, the algorithm described in Sasaki et al., “Validation and Comparison of ActiGraph Activity Monitors,” J Sci Med Sport, Vol. 14, No. 5, pp. 411-416, September 2011, which is hereby incorporated by reference for this algorithm. Many athletes and health-conscious individuals have smart watches and other measuring equipment to sense the level of activity based on movement and power generation to provide a METs value for a period of time. The movement might be based on GPS, triangulation, mechanical sensing (e.g., rotation of cycle wheel or foot falls), and assumptions based on location and gyroscope readings (e.g., lap swimming). The subjective entry by the individual will likely lead to less resolution and accuracy as compared to the other approaches.

The number of activity levels can vary and examples include 1, 2, 3, or 4 activity levels that in at least one embodiment are representative of sleeping; sitting or minimal activity; lower level activity, such as office work or cleaning; moderate level of activity such as walking; and higher levels of activity, such as running, cycling, and strenuous work. In at least one embodiment, sleeping may be combined into one activity level with minimal activity. In at least one embodiment, the higher activity level is disregarded. In at least one embodiment, the lower and moderate activity levels are consolidated. In at least one embodiment, the number of activity levels is based on the typical and/or frequent activities of the individual and how these activities might be grouped in terms of level of exertion. In a further embodiment, the individual's input or selection of activities will determine the number of activity levels for that individual and, optionally, in conjunction with the number of days to collect the baseline data, for example the longer the raw data (or baseline) collection, the more discrete activity levels may have corresponding vital-sign data to populate the time window and activity bins.

After collecting and separating the raw data, the method determines the baseline data (or baseline characteristics), 230. In at least one embodiment, the baseline data is determined by the processor 320 running software (i.e., baseline module 323). In at least one embodiment, the baseline data is determined for as many time window and activity bins as the collected raw data supports. In at least one embodiment, the baseline data includes a baseline mean and a baseline standard deviation for the vital-sign measurements, and a threshold to classify future vital signs as normal or aberrant based on how they compare to the threshold. In an alternative embodiment, the baseline mean is replaced by a baseline median. In at least one further embodiment for the baseline data, the threshold is a threshold range or a combination of a low threshold and a high threshold such that a vital-sign measurement that results in a score that is lower than the low threshold or higher than the high threshold is considered aberrant. In at least one embodiment, the baseline data is stored in the memory 321.

The baseline mean (or median) and the baseline standard deviation are statistically determined for each baseline time window (e.g., calculating using the baseline time window or the expanded time window) and activity bin for which there is vital-sign data available. The mean and standard deviation are taken across the vital-sign data of the baseline time window (or optionally the expanded time window). In at least one embodiment, when multiple days have vital-sign data for a bin for a particular baseline time window, then the raw data for each of those days are combined together.

In at least one embodiment, the threshold(s) for determining whether a particular vital-sign score is aberrant or normal is based on the vital-sign scores for all baseline time window and activity bins collected. For each time window and activity bin for each day present in the baseline period, a vital-sign score is calculated, for example to create a vital-sign scores set. In at least one embodiment, the vital-sign score S is

$$S ( {t}_{i} , {a}_{j} ) = \frac{V S ( {t}_{i} , {a}_{j} ) - m ( {t}_{i} , {a}_{j} )}{S D ( {t}_{i} , {a}_{j} )}$$

where tᵢ is the time window i in a day (or time of day), aⱼ is the activity level j, VS is the vital-sign measurement based on vital-sign data, m is the baseline mean, and SD is the baseline standard deviation. In at least one embodiment, the baseline mean and standard deviation are for an expanded window w of time around the base time window of the vital-sign, as illustrated in the equation below, where w is the expanded time window (e.g., 1, 2, 3, or 4 hours)

$$S ( {t}_{i} , {a}_{j} ) = \frac{V S ( {t}_{i} , {a}_{j} ) - m ( {t}_{i} \pm w , {a}_{j} )}{S D ( {t}_{i} \pm w , {a}_{j} )}$$

In at least one embodiment, the conversion of the vital-sign data into a vital-sign score normalizes the data across the variances from circadian rhythms. When all the vital-sign scores are added together the mean value will be zero and the standard deviation will be 1 by definition. The threshold(s) is used for comparison of future vital-sign monitoring.

The set of vital-sign scores is used to create a distribution (e.g., Weibull distribution) or distribution curve. In at least one embodiment, the entirety of the vital-sign scores is viewed as the distribution or distribution curve to determine where a lower threshold percentage or number and a higher threshold percentage or number is present in the distribution. For example, the threshold percentages might be 5% and 95% (although other percentages may be used such as between 1% and 6%, 1% or 4% depending on the level of false positive aberrant vital-signs willing to be accepted) or the X^{th} vital-sign score from the left and the X^{th} vital-sign score from the right, where the X^{th} number may correlate to the desired percentage. Using threshold percentages of 5% and 95% will lead to approximately a 10% false positive level over time (i.e., the number/percentage of vital-sign scores excluded by the two thresholds added together). Or alternatively, a threshold multiple of a standard deviation for the vital-sign score set is used. The threshold multiple might be between 1 and 2.5 with or without the end points, 1.75, or 2. In at least one further embodiment to the threshold embodiments, the lower threshold and the higher threshold are set using predetermined cutoffs such that one threshold may classify more vital-sign scores as aberrant than the other threshold. The threshold determination may be impacted by concerns of current infection rates versus the level of acceptable false positives.

In an alternative embodiment, the threshold(s) are determined for each time window and activity bin across the day using the same approach as above resulting in a number of threshold ranges or pairs of low/high thresholds to match the number of time window and activity bins.

C. Monitoring Phase

The monitoring phase of the vital-sign data begins with collection of ongoing vital-sign data and activity information for the individual, 240. The collecting ongoing vital-sign data is similar to the collecting baseline vital-sign data and as such, in at least one embodiment, the vital-sign module 310 provides the vital-sign data and the activity level module 315 provides the activity information to the processor 320 and/or the memory 321. Examples of the length of the monitoring time window is 15 minutes, 30 minutes, 45 minutes, 60 minutes, 90 minutes, 120 minutes, 150 minutes, 180 minutes, or the length of the baseline time window. The statistical analysis and correlation of the time windows are easier when the baseline time window and the monitoring time window have the same length. In an alternative embodiment, when the monitoring time window and the baseline time window have different lengths, the time windows are normalized to each other. In at least one embodiment as part of the collection of the vital-sign data, the vital-sign data is smoothed by averaging the measurements over multiple measurements, for example, if a heart rate is measured every second, then averaging the heart rates measured over 5 seconds to produce up to 360 data points per thirty minutes while smoothing the vital-sign data, for example, using a moving average over a window such as 10 or 15 minutes, and reducing the impact of any outlier vital-sign measurements.

Based on the time window that the ongoing vital-sign data corresponds to, selecting an ongoing time window and activity bin using the activity information for that monitoring time window, 250. The processor 320 (i.e., the selection module 325 or the classification module 322) uses the vital-sign data to match it to the appropriate time window and activity bin. This selection occurs similar to the matching of the raw data to bins in the baseline phase. In an alternative embodiment, when a monitoring time window includes different levels of activity that would be assigned to different bins, subdividing (or separating or segregating) the vital-sign data for that monitoring time window based on the corresponding activity levels and calculating vital-sign scores for each bin with associated vital-sign data. In a further embodiment, the threshold amount used for deciding if there is sufficient vital-sign data in a particular monitoring time window is used to determine if there is sufficient vital-sign data for the bin.

In a further embodiment, when a corresponding baseline time window and activity bin does not have baseline data, then using the prior time window and activity bin and/or the following time window and activity bin. If both before and after bins are used, then in at least one embodiment averaging the baseline vital-sign data. In a further embodiment, a bin (or an average of bins) from a neighboring time window(s) within two, three or four time windows is used.

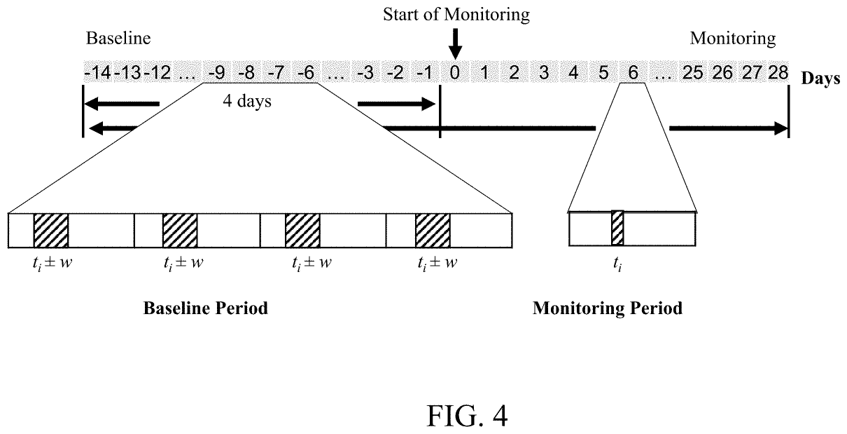

The vital-sign score is calculated based on the vital-sign data for the monitoring time window compared to the baseline data, 260. In at least one embodiment, the processor 320 running software (i.e., score calculation module 326, which in at least one embodiment is used in the baseline phase) performs the score calculation. FIG. 4 illustrates an example of heart rate normalization where there was a controlled infection, which could be replaced by the start of monitoring on Day 0, and how the baseline data is correlated to the ongoing monitoring data. In at least one embodiment, the baseline data includes the baseline mean and standard deviation for the corresponding time window and activity bin corresponding to the monitoring time window and activity bin. In at least one embodiment, the vital-sign score is

$$S ( {t}_{i} , {a}_{j} ) = \frac{V S ( {t}_{i} , {a}_{j} ) - m ( {t}_{i} , {a}_{j} )}{S D ( {t}_{i} , {a}_{j} )}$$

where tᵢ is the monitoring time window i in a day, aⱼ is the activity level j, VS is the vital-sign measurement based on vital-sign data, m is the baseline mean, and SD is the baseline standard deviation. In at least one embodiment, the vital-sign measurement for the monitoring time window is the mean for that monitoring time window. In an alternative embodiment, the vital-sign measurement for the monitoring time window is the mean for the expanded time window w around the monitoring time window as illustrated in the following equation:

$$S ( {t}_{i} , {a}_{j} ) = \frac{V S ( {t}_{i} , {a}_{j} ) - m ( {t}_{i} \pm w , {a}_{j} )}{S D ( {t}_{i} \pm w , {a}_{j} )}$$

In at least one embodiment, the vital-sign score is stored in the memory 321.

The vital-sign score is compared to the threshold range to classify the vital-sign score as normal or aberrant, 270. In at least one embodiment, the processor 320 (i.e., the aberrant module 327) performs the comparison and determination. If the vital-sign score falls within the threshold range or between the low and high thresholds, then the vital-sign score is normal; otherwise, the vital-sign score is aberrant. When a time window has multiple activity levels with vital-sign data, then subdividing the time window based on the activity levels before calculating a score for each activity level in that time window using the corresponding bin. The total number of aberrant or normal determinations for that monitoring time window will be between 1 and the number of activity levels. In at least one embodiment, the number of normal or aberrant determinations are added together or alternatively are averaged over the number of such activity levels.

Next, a probability is calculated whether for the individual has an adverse condition, such as an infection, 280. In at least one embodiment, the processor 320 running software (i.e., probability module 328) performs the probability calculation. The probability is calculated using whether the vital-sign score(s) was normal or aberrant, an aberrant vital-sign probability for a healthy individual, an aberrant vital-sign probability for an infected individual, and after the first probability calculation, the previous probability. Or alternatively, the probability is calculated using whether the vital-sign score is aberrant or normal, a ratio that an aberrant vital-sign would happen in a healthy person as compared to an infected person. In at least one embodiment, the probability uses a Bayesian probability, for example

$${p}_{t} = \frac{1}{1 + {( \frac{{p}_{h}}{{p}_{i}} )}^{n} {( \frac{1 - {p}_{h}}{1 - {p}_{i}} )}^{k} ( \frac{1}{{p}_{t - 1}} - 1 )}$$

where pₕ is a probability to observe an aberrant vital-sign in a healthy subject (or healthy individual), pᵢ is a probability to observe an aberrant vital-sign in an infected subject (or an infected individual), t is the monitoring time window, and n and k, respectfully, are the number of aberrant and normal vital-signs for the individual observed in the monitoring time window (i.e., the time window between time t−1 and time t). In at least one embodiment, the pₕ and pᵢ are preset. In at least one embodiment, the pₕ and pᵢ are computed to maximize Shannon's entropy. In a further or alternative embodiment, a ratio of pₕ and pᵢ is used as a parameter to optimize the probability model. The first time using the probability calculation, the value for pₜ₋₁ may be set between zero and one and not including the end points, between 0.05 and 0.5, 0.1, an arbitrarily small number less than 0.25, or an estimation of the probability that the individual may be infected, for example based on recent risk of infection or recent infection exposure. In at least one embodiment, the probabilities are stored in memory 321.

In a further embodiment, the probability is adjusted based on reducing the impact of the previous probability over time by using a forgetting (or sunset) multiplier α such that the probability reduces to zero or at least a very small probability when the number of aberrant vital-signs are zero or at least a very small number over time, for example about 5 days although other time horizons may be used. The forgetting multiplier α may be in the range of 0.9 to 1, 0.95 to 1, 0.988, 0.99, 0.996, smaller than 1, or any number in these ranges. In at least one embodiment, the probability becomes

$${p}_{t} = \frac{1}{1 + {( \frac{{p}_{h}}{{p}_{i}} )}^{n} {( \frac{1 - {p}_{h}}{1 - {p}_{i}} )}^{k} ( \frac{1}{α * {p}_{t - 1}} - 1 )}$$

In at least one embodiment, a ratio of pᵢ/pₕ of 1.7 was found to work with a forgetting multiplier α of 0.988.

In a further embodiment when there is no vital-sign data and/or activity information for a current time window and a probability is desired, the method infers a probability based on at least one prior probability. One approach to inferring the current probability is to maintain the probability level for a predetermined number of monitoring time windows, where the number may be in a range of 1 to 16, a range of 1 to 12, a range of 4 to 10, and the ranges with or without one or the other end point. A second approach is to steadily decrease the probability for each monitoring time window where pₜ equals a decreased amount of pₜ₋₁ for example using the forgetting multiplier α. A third approach is to use the trend over the previous Y number of monitoring time windows where Y equals the number of monitoring time windows with no vital-sign data plus 1, or, alternatively, the trend is over the preceding two or three monitoring time windows. A further alternative embodiment, would use the average for the aberrant and normal values over the preceding monitoring time windows. A fourth approach is to arbitrarily classify the monitoring time window as having a normal (e.g., setting k=1) and/or an aberrant (e.g., setting n=1) vital-sign score, which in at least one further embodiment would include providing a probability range (e.g., determining a high probability by setting n to 1 and a low probability by setting k to 1) that would disappear over time. In a further embodiment, two to four of the approaches are used in some combination with each other to provide an estimated current probability. In a further embodiment, alerting the individual or another person if no vital-sign data is present for a predetermined time.

The above-described probability approaches are examples of a probability model.

The calculated probability is provided to the individual (and/or another person) or an entity, 290. The information may be displayed, for example on a display 390 by the processor 320, as a probability or converted to an infection score, for example as a number, a gauge or other graphical representation. Alternatively, or in addition, the probability is transmitted to an external computer and/or to another person's display. In a further embodiment, alerting the individual (and/or another person) when the probability exceeds a probability threshold allows the individual, for example, to seek medical assistance, isolate, or take other action. If another person and/or entity is alerted, then a work schedule may be adjusted to consider the potential unavailability of the individual. In a further embodiment, the probability threshold is set at a level appropriate for infection frequency in an area in which the person is present such that the probability threshold is the lower of 50% or 100% minus a current infection rate.

In a further embodiment to the above embodiments discussed in connection with FIG. 2, monitoring at least one more vital sign occurs. For each additional vital sign being monitored, establishing the baseline data and then monitoring that vital sign includes calculating a probability for that additional vital sign. The two or more probabilities are combined together either by averaging or weighting the probabilities based on which vital signs are being used. In a further embodiment, multiple combinations may be tracked when a change of probability weights provide improved accuracy for different pre-symptomatic adverse conditions.

Further to any of the method embodiments, the method collects baseline and monitoring vital sign data and activity information for predetermined time windows during the course of the day. For example, the time windows could be spaced from each other by a few hours or at select times in the morning, midday, and evening. The probability model would use the prior time window for the previous probability.

In at least one embodiment, the vital sign being used is the individual's heart rate. In an alternative embodiment, the vital sign(s) is selected from a group including heart rate, pulse rate, blood pressure, or blood oxygen saturation measured directly or indirectly estimated. In alternative embodiment, the vital sign is respiratory rate. In another alternative embodiment, the vital sign(s) is selected from a group including heart rate, respiratory rate, pulse rate, blood pressure, or blood oxygen saturation measured directly or indirectly estimated. In further alternative embodiments, one or more vital signs measured of the individual is selected from: respiratory rate (RR), oxygen saturation (SaO₂ or SpO₂), electroencephalography (EEG), electrocardiogram (ECG or EKG), heart rate (FIR), skin temperature, and combinations thereof. In one embodiment, the vital-sign data measured in the subject using the wireless wearable device are selected from heart rate, skin temperature, ECG or EKG, EEG, and combinations thereof. One or more vital signs measured by other sensors attached to the individual may be selected from: cardiac output index CO/m² (CI), systematic pressures (systematic systolic arterial pressure (SSAP), systematic diastolic arterial pressure (SDAP), systematic mean arterial pressure (SMAP), central venous pressure (CVP), pulmonary pressures (pulmonary systolic arterial pressure (PSAP), pulmonary diastolic arterial pressure (PDAP), pulmonary mean arterial pressure (PMAP), oxygen saturation in the lung artery (svO₂), outcoming carbon dioxide (ETCO₂), ingoing oxygen (FIO₂), fluid balance (ingoing and outcoming fluids), and combinations thereof. Based on this disclosure, these vital-signs may be used.

Although one processor 320 and one memory 321 are discussed in connection with the above-described method embodiments, the processor and/or the memory are representative of processors/memories that can be distributed across one device or multiple devices depending on a particular implementation. The described modules may take a variety of forms depending on the particular implementation. Based on this disclosure, it should be understood that the varying method embodiments for particular steps below those illustrated in FIG. 2 may or may not be combined with other particular steps from other flowchart steps.

D. System

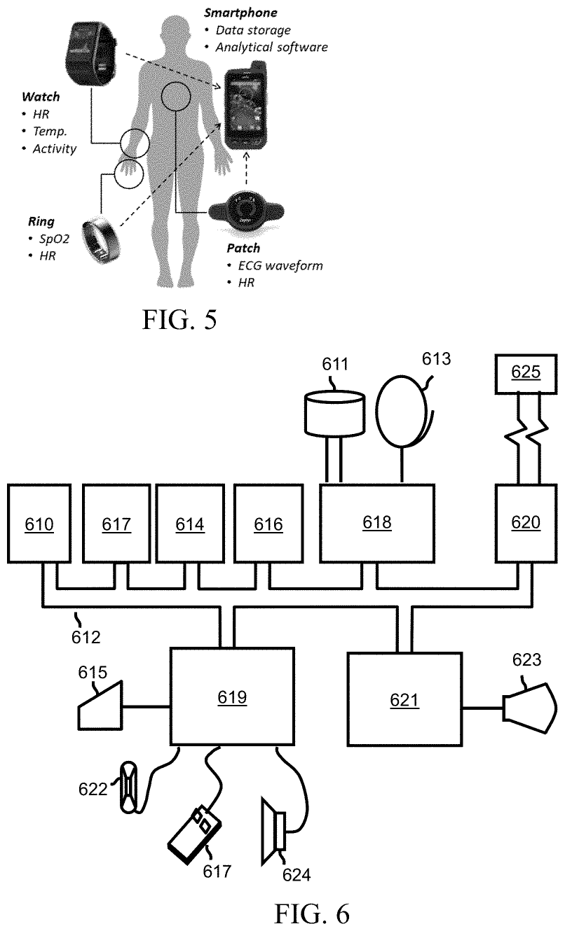

A variety of devices and systems may be used to perform the above-described methods including the use of a wearable device(s) (or body worn vital-sign measurement device(s)) such as a smart watch, a smart ring, a patch, a fitness tracker, an ear piece, a bandage (discussed later), or other wearable sensing device, each of which then may be in wireless communication (e.g., over a wireless network or, alternatively, by a hardwire connection as might occur during a synchronization between devices) with a computing device (or alternatively a wireless mobile device), which may be a smart phone, a tablet, a computer or other computing device as illustrated in FIG. 5. In a further embodiment, the computing device is in turn in communication with a server, or alternatively the wearable device communicates with the server. In an alternative embodiment, the wearable device performs all the method steps. In an alternative embodiment, the initial collection of baseline vital sign data is done using temporarily affixed monitoring equipment at different times of day and after (or during) different levels of activity such as may occur in a laboratory or medical office setting. Depending on the particular implementation, one or more devices may perform the method.

At least one wearable device functions to continuously measure one or more vital-signs in the individual. The wearable device includes one or more sensors that continuously measures vital-sign data from an individual and can track or provide activity information, for example derived from a 3-axis accelerometer output, METs, and/or user input.

In a separate embodiment, the wireless wearable device used to measure vital-sign data in the individual can be implemented as a conventional bandage (or other adhesive mechanism) attached to the individual's body without complicated or extensive preparation of the individual. Such a device is extremely simple and economical of construction, it may be utilized as a one-time use, throwaway device which permits high mobility of the individual while yet providing continuous monitoring of the required vitals sign data. The device continually monitors one or more of the individual's vital-sign data, with reduced susceptibility to noise and subject movement, and relative insensitivity to placement of the device on the individual. The device also would not need recalibration while in use.

In a further embodiment, at least one wireless mobile device is in wireless communication with the wearable device(s) via a wireless network and the wireless mobile device functions to collect and store the continuously measured vital-sign data from the individual. The wireless mobile device includes one or more analytic software applications (apps) platform capable of processing continuous vital-sign data measured in real-time from the individual and in a further embodiment, perform the above-described methods. In at least one embodiment, the wireless mobile device is a cellular telephone (smartphone) having a wireless transceiver adapted to communicate with at least one computing network via a wireless network; and a 3-axis (or 2-axis) accelerometer or other capability to monitor the activity level of the individual by itself or in conjunction with other devices.

At least one computing network employed in accordance with at least one embodiment of the invention is in communication with the wearable device, the wireless mobile device and the wireless network, wherein the computing network compares continuously measured vital-sign data from the individual with baseline vital-sign data for the individual to identify deviations in one or more of the vital-signs of the individual that are indicative of a pre-symptomatic infection, early stage infection or symptomatic infection in the individual.

Referring now to FIG. 6, a representative hardware environment for practicing at least one embodiment is depicted. This schematic drawing illustrates a hardware configuration of information handling/computer system components, which in a further embodiment may be representative of a smart phone architecture other than the I/O adapter and some peripheral components, in accordance with at least one embodiment of the invention. The illustrated system includes at least one processor or central processing unit (CPU) 610 (for simplicity, one CPU is illustrated). The CPU(s) 610 is interconnected with a system bus 612 to various devices such as a random-access memory (RAM) 614, a read-only memory (ROM) 616, an internal non-volatile storage 617, and an input/output (I/O) adapter 618. The RAM 614, the ROM 616, and the storage 617 are examples of the memory 321. The I/O adapter 618 may connect to peripheral devices, such as disk (or storage) units 611 and tape drives 613, or other program storage devices that are readable by the system. The system can read the stored instructions follow these instructions to execute the methodology of at least one embodiment of the invention. The system further includes a user interface adapter 619 that connects a (virtual) keyboard 615, a mouse (or virtual cursor) 617, a speaker 624, a microphone 622, and/or other user interface devices such as a touch screen device (not shown) to the bus 612 to gather user input. Additionally, a communication adapter 620 connects the bus 612 to a data processing network 625 (e.g., wireless network, cellular network, or broadband connection), and a display adapter 621 connects the bus 612 to a display device 623 which may be built into the system or embodied as an output device such as a monitor, printer, or transmitter, for example.

In at least one implementation, the system includes one or more wireless networks which functions to transmit the vital-sign data measured for the individual wearing the wearable device to a wireless mobile device, a computing device, or a server. Alternatively, the measured vital-sign data is transmitted via existing transceivers using, for example, Zigbee, Bluetooth, or WiFi. The frequencies for a Medical Body Area Network (MBAN) network (2360-2400 MHz) may be used and can be the aeronautical mobile telemetry (AMT) frequency or an encrypted military frequency, for federal radio location tasks, and by amateur radio operators. The bandwidths cover 2360-2400 MHz, 2300-2305 MHz and 2395-2400 MHz, 2400-2483.5 MHz, or 5150-5250 MHz and reside next to those now used by Bluetooth devices. In addition, the modifications proposed by industry would use the 2390-2400 MHz range as a secondary MBAN when the primary frequencies interfere with aeronautical industry communications. By allocating spectrum for medical sensors, individuals can avoid having wireless dead zones interrupt their transmission of vital-sign data to other people or entities, for example insurance companies, aggregation collection sites, medical facilities, and doctors. This spectrum, previously reserved for commercial test pilots, could be used instead. It is likely that most of the world would have ability to use the 2,360 to 2,390 MHz band while avoiding aeronautical receive locations.

In at least one embodiment, the wireless network, wearable devices, wireless devices and computing networks will contain required security. In one embodiment, the system may use 64-bit encryption as well as 128-bit AES encryption. The system may implement multiple layers of security measures to control access to mission-critical systems and networks, which are often the targets that an attacker attempts to gain unauthorized access to by compromising a wireless network and using it as an attack path or vector in to an organizational network such as a hospital network where the target systems reside. To defend the target environment, multiple security measures may be implemented so that if one measure is defeated by an attacker, additional measures and layers of security remain to protect the target environment. Measures such as separation of wireless and wired network segments, strong device and user authentication methods, filtering of traffic based on addresses and protocols, securing end-points/stations from unauthorized access, and monitoring and intrusion detection on the wireless and wired segments are examples of multiple layers of defense that can be employed to achieve a defense-in-depth design. The wireless networks and wired networks should not be directly connected if possible. For example, a wireless environmental sensing Low Rate Wireless Personal Area Network (LR-WPAN) or equipment monitoring LR-WPAN should not have direct connectivity to the wired healthcare network, but instead be separated by a device such as a firewall, bastion host, or security gateway to establish a security perimeter that can more effectively isolate, segment, and control traffic flows between them. Security features at the upper layer standards and the IEEE lower layer standards are enabled. Both standards have security services defined in their specifications. Having security defined at both the higher and lower layers of the protocol stack creates a stronger security solution. Source node authentication is implemented to cryptographically verify the identity of a transmitting node. Although a shared network key will provide a security check for packets utilizing the network, source node authentication can be used by the destination device to verify the identity of the source device. To authenticate a source device, a link key (end-to-end crypto key) must be generated and used. This key is unique to a pair of devices that are communicating with each other and is derived from their respective master keys. This is equivalent to the concept of a shared secret or unique session key that is derived between two entities in order secure data transmitted between them.

A study was conducted with continuous vital-sign monitoring via non-invasive, wireless wearable devices in a controlled human infection study, providing a range of physiological measurements, including heart rate, skin temperature, and electrocardiogram (ECG), and 3-axis accelerometer data. Early-stage infection is characterized by distinct changes in vital-sign patterns that was detected by real-time monitoring. Using malaria as a surrogate model for infection, the method utilized data from a CHMI study, in which human subjects were intentionally infected with malaria in a controlled clinical environment, to measure continuous vital-sign data throughout the course of a malaria infection. Commercial off-the-shelf (COTS) wireless wearable devices based on multiple criteria, including accuracy, usability, and hardware architecture were employed in accordance with the invented system. The wearable devices were worn by CHMI study subjects, who otherwise went about their normal daily activities and a wide range of vital-sign data was collected over a four-week period that included baseline, incubation (pre-symptomatic infection or “aberrant”), and parasitemic (symptomatic infection) periods. The incubation and parasitemic periods would fall into the monitoring period. Using the vital-sign data collected in the CHMI study, multiple approaches were employed, involving signal processing and pattern recognition of time-series data and including autoregressive (AR) models, to detect vital-sign deviations indicative of infection. Deviations in vital-sign measures of the subject were identified that are most indicative/predictive of infection, and a working model for indicating/predicting the likelihood of infection directly from continuous vital-sign data in one or more subjects. Analytical tools were developed for characterizing changes in vital-sign measurements in the context of baseline (“normal”) and disease (sub-clinical and clinical infection) conditions to make a health status assessment that indicates/predicts the likelihood of infection in the subject at a given time point. A range of regression analysis methods were employed to characterize physiological changes associated with infection and develop a predictive model that estimates the likelihood of infection at any given time point.

In at least one embodiment, the system and method provide an indicative/predictive model for detecting infection in one or more subjects. Previous approaches for detecting early-stage infection from continuous vital-sign data monitoring of the subject are impacted by data being noisy and complex when measured in everyday, ambulatory conditions, and are subject to signal fluctuations due to technical factors related to the sensors themselves, as well as physiological factors (an individual's behavior, activity, circadian rhythm, etc.). To determine reliable patterns associated with infection, analysis of real-time vital-sign data must be able to account for the technical and physiological sources of noise and data variation in baseline, non-infection conditions.

As discussed below, at least one implementation of the system and method have received validation using controlled human infection models. To assess the accuracy of real-time detection of infection status in a specific individual, these models were employed in accordance with at least one embodiment in a controlled infection setting, where the exact times of exposure and infection are known. Typical clinical studies, which rely on self-reporting or scheduled clinical checkups to assess infection, cannot capture the time of exposure accurately enough to detect an infection in real time. Controlled human infection studies, in which subjects are infected in a controlled laboratory environment, provide an alternative means for characterizing the exact course of an infection.

E. Malaria Studies

The following discussion includes citation to different references identified at the end of the specification by the use of brackets.

An implementation of the invention was found to provide a solution to unaddressed challenges known in the prior art by using non-invasive wireless wearable devices to collect continuous vital-sign data from participants in controlled human infection studies and by employing sophisticated analytical methods for early identification of potential vital-sign patterns indicative of infection. Using computational methods, including multiple approaches for signal processing and pattern recognition of time-series data, deviations in vital-sign patterns in subjects were identified that are associated with infection in the subjects, which can be used to detect infection status in subjects in real time. In these studies, malaria was used as a surrogate model for infection because CHMI is a well-characterized challenge model regularly used in malaria vaccine clinical trials [1]. Furthermore, the clinical manifestations of malaria have been well studied in humans, and common symptoms, such as periodic high fevers, may be detectable by the wireless wearable devices of the developed system and method.

Malaria is caused by infection of a mosquito-borne protozoan parasite of the Plasmodium family, usually Plasmodium falciparum or Plasmodium vivax. Following a bite by an infected mosquito, the initial incubation stage of the infection is asymptomatic and lasts, on average, ten days before progressing to the blood stage, which is characterized by blood parasitemia and fever and which leads to more severe symptoms unless treated [2]. Animal studies have shown that the Plasmodium parasites travel from the site of the mosquito bite in the epidermis, through the blood stream, and into the liver within 24-72 hours post-infection. Within the liver, the parasites infect liver cells for a period of six to nine days, before emerging from the liver in the blood stage of the infection. In the blood, the parasite rapidly reproduces within the host's red blood cells, causing parasitemia and anemia, along with a range of more severe symptoms, such as paroxysms, joint and muscle pain, and high fever. Because the incubation stage is largely asymptomatic, relatively little is known about its pathophysiology.

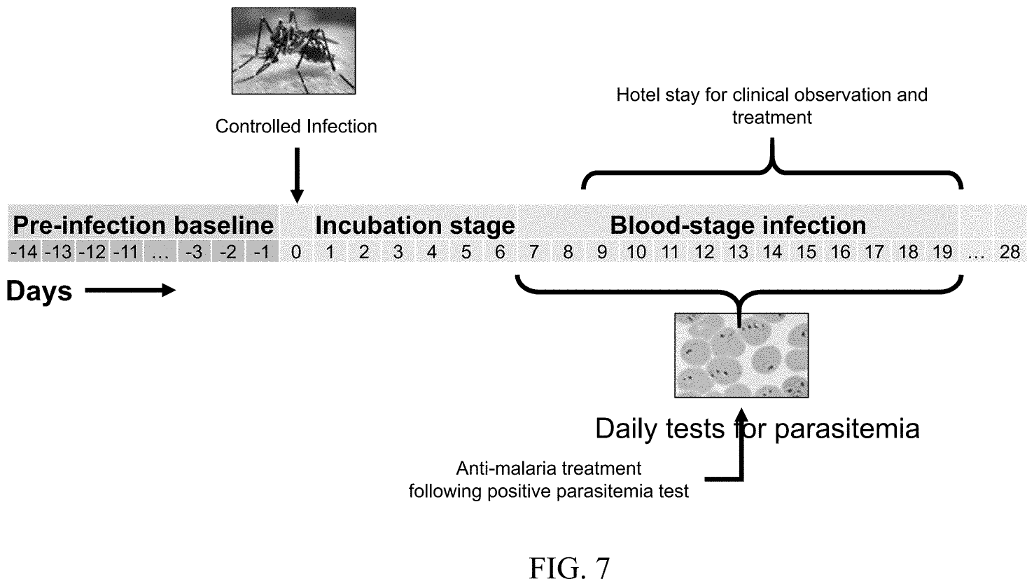

The original laboratory-controlled CHMI study, using mosquitoes infected with laboratory cultured P. falciparum, was conducted as a collaboration between the U.S. Army, U.S. Navy, and National Institutes of Health in 1986 [3]. Since that seminal study, over 1,000 volunteers have been challenged in CHMI studies at the Walter Reed Army Institute of Research (WRAIR) and the Naval Medical Research Command (NMRC). In a typical CHMI study, 10-15 subjects are recruited and baseline immunological measurements are collected at least seven days prior to the challenge. Study subjects are screened for prior exposure to malaria. During the challenge, subjects are exposed to five Plasmodium-infected mosquitoes over a period of 5 minutes to ensure that they receive a sufficient dose of sporozoites for infection. From post-challenge Day 5, subjects receive daily blood-smear assays to test for blood-stage parasitemia until either parasitemia is detected or the study is concluded (Day 28). From Days 9 to 19, when the blood-stage infection is most likely to emerge, subjects are required to spend their nights in a controlled environment for clinical observation of potential malaria symptoms. Once parasitemia is detected, clinicians immediately administered an FDA-approved antimalarial drug that rapidly clears the infection. Vital-signal data from CHMI studies were used for model development and are typically designed as illustrated in FIG. 7. Subjects were enrolled two weeks prior to challenge to obtain baseline pre-infection vital-sign measurements. Following controlled infection on Day 0, the asymptomatic incubation stage of malaria typically lasts six to nine days, followed by the symptomatic blood-stage infection. Parasitemia typically coincides with the emergence of clinical malaria symptoms.

In a review of 17 CHMI studies [4], Epstein et al. identified several common features associated with controlled malaria infection. First, the period between challenge and the onset of symptoms (incubation period) tended to slightly precede the period between challenge and the onset of parasitemia, as detected by blood smear. Approximately 60% of the subjects reported malaria symptoms at least 12 hours prior to detection of parasitemia, and 30% reported symptoms on the same day that parasitemia was detected. Given that newer diagnostic measures, such as quantitative real-time PCR (qPCR), can detect parasitemia 1-2 days earlier than blood smear [5], a positive parasitemia test generally precedes noticeable malaria symptoms. Epstein et al. also noted that body temperatures measured at post-challenge Day 10 were greater than 37.5° C. for 80% of subjects, greater than 38.0° C. for 60%, and greater than 40° C. for 5%. The reported symptoms were classified as “mild” to “moderate” in ˜80% of subjects, although 20% reported having “severe” symptoms, based on criteria defined by the FDA. The most common symptoms were fatigue, headache, malaise, chills, and myalgia, lasting from two to four days. Overall, these findings suggested that the CHMI protocol reliably produces early-stage malaria infection, and that rapid diagnosis and treatment prevents the onset of more severe malaria illness typically seen in natural infections.

In malaria, the incubation stage of infection lacks overt symptoms and there is no FDA-approved diagnostic assay for detecting its presence. As such, little is known about the physiological changes associated with this early stage of infection. Furthermore, given the duration (>2 weeks) and variability of the malaria infection process, it is infeasible to continuously monitor vital-signs using traditional sensors that are physically tethered to stationary equipment, because this would require subjects to be confined to a small space for a period of multiple weeks. Non-invasive wearable devices are the only feasible alternative for measuring continuous vital-sign data for the duration of a typical infection.

TABLE 1
CHMI studies at WRAIR/NMRC

| Name | Type | Lab | Date | Description |
|---|---|---|---|---|
| RTS, S | Vaccine | WRAIR | 2018 | Assess the efficacy of the |
| Clinical | efficacy | | | RTS, S malaria vaccine in |
| Trial | | | | several regimens |
| Bridging | Infection | WRAIR | 2019 | Measure infectivity rates for |
| Study | only | | | new lot of 3D7 strain of P. |
| | | | | falciparum |
| NavOx | Vaccine | NMRC | 2019 | Assess the efficacy of the |
| Clinical | efficacy | | | NavOx malaria vaccine |
| Trial | | | | candidate |
| Dose | Infection | NMRC | 2019 | Optimize sporozoite doses |
| Optimization | only | | | for NF135.C10 strain of P. |
| | | | | falciparum |

In addition to the essential role that WRAIR/NMRC played in the development of the CHMI challenge model, the WRAIR insectary continues to be the largest source of clinical-grade malaria parasites for use in CHMI studies worldwide. As a result, several CHMI studies are conducted every year, on-site, in Silver Spring, Md. Table 1 above lists a selection of CHMI studies that have been performed by WRAIR/NMRC and were compatible for use in a wearable-device study. These CHMI studies fall into two categories: infection-only studies in which the rate of infection and the number of subjects infected at a given challenge dose were measured, and vaccine-efficacy studies in which the number of subjects protected from a challenge following immunization using a candidate malaria vaccine was assessed.

Recent advances in sensor miniaturization have led to the emergence of highly ergonomic, non-invasive, commercially available wearable devices that can measure a range of vital-sign data, including heart rate, skin temperature, ECG, blood oxygen saturation, and 3-axis accelerometer data. Typically, these devices require daily or weekly re-charging and are tethered, wirelessly via Bluetooth, to an Android or iOS smartphone. FIG. 5 illustrates a representative suite of wearable device types that measure a variety of vital-sign data, wirelessly tethered to a smartphone to be considered in this study. The utility of non-invasive wearable devices for collecting data under every day, ambulatory conditions holds enormous potential for these sensors to provide the capability for real-time physiological monitoring of infections. However, two major gaps in the understanding of this technology needed to be addressed. First, it is unclear whether sensors on wearable devices have sufficient accuracy, compared to clinic-grade medical devices, to reliably capture physiological changes associated with infection. Second, particularly for pre-symptomatic or pre-clinical infections, it is not known precisely what those physiological changes and corresponding vital-sign changes are, and whether they are predictive of infection.

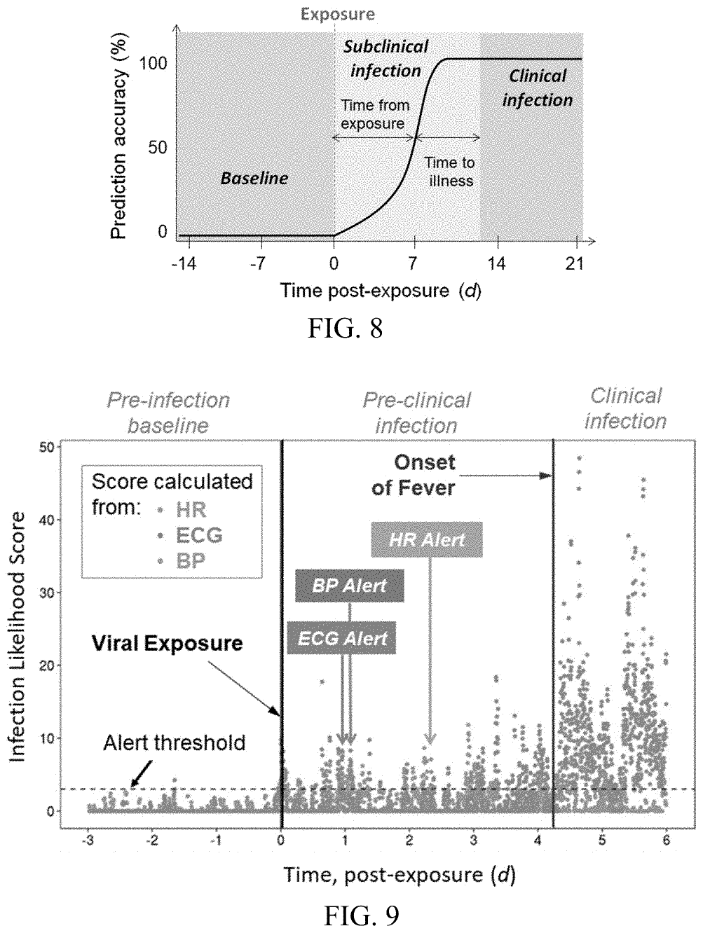

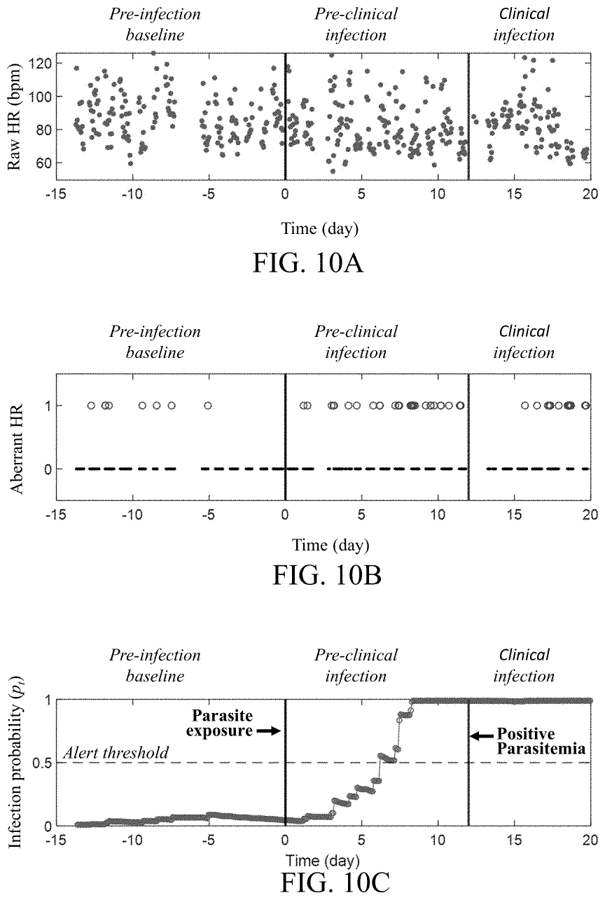

The value of an early-warning system arises from its ability to provide a reliable and accurate prediction of infection prior to the manifestation of clinical symptoms of illness. Because the time course of an infection can vary between individuals, it is important to implement an evaluation strategy that measures the accuracy of predictive models based on both time-from-exposure, defined as the time between exposure and a positive prediction of infection, and time-to-illness, defined as the time between a positive prediction of infection and the clinical manifestation of that illness illustrated in FIG. 8. In CHMI studies, the period between the beginning of the study and pathogen exposure is defined as the “baseline,” that between exposure and the first day where parasitemia is detected as the “subclinical infection period,” and that following detection of parasitemia as the “clinical infection period.” Parasitemia is typically assessed daily using either the FDA-approved blood smear test [4], or using a newer, more sensitive qPCR-based measure [5].

One of the challenges in developing timely and accurate early warning systems is to balance the need for earliest possible detection (sensitivity) versus the need to minimize false-positive detection (specificity). The best trade-off between these opposing needs, of balancing sensitivity and specificity of alert, is provided by the sequential probability ratio test (SPRT), which has been shown to achieve this goal while requiring the smallest number of time-series samples. This is achieved at every time-series measurement by computing the likelihood ratio test for the null hypothesis (the subject is not infected) versus the alternative hypothesis (the subject is infected), and then comparing this test with pre-specified thresholds for a desired sensitivity and specificity.

Preliminary results on infection detection that arose in a research project with continuous vital-sign data in non-human primates (NHP) exposed to Ebola virus, Marburg virus, or Lassa virus has been analyzed to identify infection-associated changes in vital-sign data. NHP vital-signs were continuously monitored and recorded via sensors implanted as part of a telemetry system. The physiological measurements included ECG, body temperature, blood pressure, respiratory rate, and 3-axis accelerometer data. A preliminary analysis revealed that a number of measures showed statistical power in predicting infection before clinical symptoms, such as fever. In one study, the power of individual vital-signs was evaluated using data from seven NHPs infected with Ebola virus. Autoregressive (AR) modeling was used to calculate an infection likelihood score for three types of vital-sign data: heart rate, ECG, and blood pressure. An “alert threshold” was defined based on pre-infection baseline data and determined, for each vital-sign measure, if and when the infection likelihood score exceeded the alert threshold. FIG. 9 shows a representative example from a single NHP subject, in which the infection likelihood calculated for all three vital-signs resulted in a prediction of infection 1-2 days post-exposure and 2-3 days prior to the onset of fever. Of the 7 NHPs in this study, infection likelihood scores calculated for heart rate, ECG, and blood pressure could predict infection at least 48 hours prior to the onset of fever in six cases. These preliminary results suggest that it should be possible, based on continuous vital-sign monitoring, to develop predictive models that can detect infection prior to the clinical manifestation of symptoms.

In the multi-disciplinary effort, the inventors worked with clinical researchers at WRAIR and NRMC to collect real-time vital-sign data, using non-invasive wearable devices in a CHMI feasibility study; develop new analytical techniques to identify vital-sign patterns indicative of malaria infection; and then implement a new CHMI study to blindly evaluate the performance of our early detection algorithm. The clinical component of the study was carried out by malaria researchers at WRAIR/NMRC. The computational component of the study was carried out at the Biotechnology High Performance Computing Software Applications Institute (BHSAI).

The unique aspect of this study was that it was the first of its kind to collect and analyze continuous vital-sign data throughout the entire course of infection, from the initial exposure to its resolution. Approaches attempted by other groups include endotoxin inhalation, which induces a transient innate immune response, and vaccination using a live-attenuated vaccine, which establishes a largely non-pathologic infection. By contrast, the CHMI model results in the establishment of an infection with a well-defined progression to disease, capturing both asymptomatic pre-clinical aspects of the infection all the way to its symptomatic clinical aspects. To date, however, no human study has captured continuous vital-sign data in a controlled infection study. The data set and resulting analysis of this study will be essential to future research efforts in continuous and real-time physiological monitoring of bio-threats.

The following hypotheses together formed the conceptual basis of the approach:
pre-clinical infection, prior to the establishment of overt clinical symptoms, results in measurable physiological changes in vital-sign patterns from baseline;
continuous vital-sign data collected from prior to exposure, through the early subclinical stage of infection, can identify measurable physiological changes associated with infection; and
infection-associated physiological changes can be used to develop a predictive model that uses continuous vital-sign monitoring to make real-time assessments of the likelihood of infection.

The main hypotheses were addressed, as described above, by performing a number of technical tasks organized around the following four specific aims.

1. Collect Continuous Vital-Sign Data Using Wearable Devices from a Controlled Human Infection Study

Commercially available wearable devices were selected and then used on human subjects in the CHMI study to collect continuous vital-sign data for a duration of 4-5 weeks—from two weeks prior to exposure to one week after clearance of the infection. It is important to note that the vital-sign data was collected from untrained subjects under every day, ambulatory conditions. In this initial data collection effort, the aim was to collect as many types of vital-sign data as feasible, including heart rate, skin temperature, oxygen saturation (SpO₂), ECG, and 3-axis accelerometer data. Wireless wearable devices were selected based on multiple criteria, including accuracy, usability, and open data architecture.

All wearable devices for collecting vital-sign data in this study were intended to be non-invasive, commercially available with open architecture, and linkable to a smartphone device via a wireless Bluetooth connection. Table 2 lists several candidate devices that were either already commercially available or would be in the near future. These devices were evaluated to assess their accuracy and usability before selecting a final set of wearable devices for use in the CHMI clinical trial. Two devices—the Samsung Gear S3 and the Zephyr BioPatch—were taken as “default” devices for consideration. These two devices are described in detail and how they were used in the study.

The primary hardware component for this study was the wearable device—a commercially available smartwatch (Samsung Gear S3 or equivalent)—wirelessly linked to a standard Android smartphone. The inventors have experience developing custom data analysis applications for the previous generation Samsung Gear S2, and newer Gear S3 watches that allow recording of heart rate from the optical sensor at a sampling rate of 1 Hz, skin temperature at a sampling rate of 1 Hz, and raw 3-axis acceleration at sampling rates between 8 and 62 Hz. The data was transferred automatically via wireless Bluetooth communication to a paired Android smartphone running a custom Physiological Data Recorder (PDR) app. Data acquisition was completely automated and no user input was required. Furthermore, the watch stored the data internally until the wireless connection was reestablished. Thus, the smartphone need not be carried continuously by the subject for data collection. Gear S3 can collect data continuously for up to 30 hours on a single battery charge, with recharging taking up to 3 hours.

TABLE 2
Candidate commercially available wearable devices

| | | | Real- | | |
|---|---|---|---|---|---|
| | | | time | Data | |
| | | | com- | Storage/ | Battery |
| Device | Type | Vital-signs | patible | Transfer | Life |
| Samsung | Wrist- | 3-axis accel., HR, | Yes | 30 d/ | 30 h |
| Gear S3 | watch | skin temp. | | Bluetooth | |
| Zephyr | Chest | 3-axis accel., step | Yes | 55 h/ | 24 h |
| BioPatchHP | patch | count, ECG, HR, | | Bluetooth, | |
| | | HRV, skin temp., | | USB | |
| | | breathing rate | | | |
| SensoGram | Finger | step count, ECG, | Yes | 48 h/ | 48 h |
| SensoRing | ring | PPG, HR, HRV, BP | | Bluetooth, | |
| | | | | USB | |
| Wavelet | Wrist- | 3-axis accel., PPG, | Yes | None/ | 27 h |
| Biostrap | band | SpO2, HR, HRV, | | Bluetooth | |
| | | RR | | | |
| Empatica | Wrist- | 3-axis accel., PPG, | Yes | 60 h/ | 36 h |
| | band | HR, skin temp. | | Bluetooth, | |
| | | | | USB | |
| Everion | Arm- | 3-axis accel., PPG, | Yes | None/ | 30 h |
| Biovotion | band | HR, SpO2, Skin | | Bluetooth | |
| | | Temp. | | | |

BP, blood pressure;
ECG, electrocardiogram;
HR, heart rate;
HRV, heart rate variability;
PPG, photoplethysmography;
temp., temperature.

Real-time ECG measurements were obtained, using the BioPatch Monitoring Device Human Performance (Zephyr Technology Corp. (Medtronic), Annapolis, Md.). The Zephyr BioPatch is a lightweight (32 g) multiple-use sensor with disposable adhesive chest electrodes. The device collected waveform data via a single-lead ECG (at a sampling rate of up to 1 kHz), 3-axis accelerometry (50 Hz), and breathing rate (25 Hz). The Zephyr BioPatch transmitted live vital-sign data via Bluetooth, or store internally up to 55 hours of data for later download. The battery life is about 24 hours for live streaming and 35 hours for internal logging, with full charging requiring up to 3 hours. Single-use ECG monitors (ZioXT; iRhythm, San Francisco, Calif.), which are alternatives to the Zephyr BioPatch, provided maximum comfort and ease of use. However, their lack of wireless capability prevents or at least impacts the ultimate goal, which is the development of a real-time system.

The development of any continuous physiological monitoring capabilities based on vital-sign measurements from wearable devices critically depend on the accuracy of the measurements. For each vital-sign, paired recordings of data were collected from the candidate wearable device and a gold-standard device to ensure that the accuracy of the test device was either comparable to clinical measurements or meets the threshold deemed to be sufficient to detect subclinical, pre-symptomatic infection. Accuracy was measured in terms of the root mean squared deviation between the wearable device and the corresponding gold-standard device. For example, as the gold-standard device, the Polar H7 chest strap heart-rate monitor (Polar Electro Inc., Bethpage, N.Y.) was used for heart rate, and the wireless iButton temperature sensor (OnSolution Ltd., Baulkham Hills, Australia) was used for skin temperature.

Preliminary data from research, or existing published data, was used to determine thresholds for the accuracy needed to detect infection by comparing the change of a particular vital-sign between baseline and infection conditions. For example, in the NHP study involving Ebola infection (described above), the heart rate was found to increase by 5-10 beats per minute (bpm) at 24 to 48 hours prior to onset of fever, suggesting that an accuracy threshold of 10 bpm for heart rate may be sufficient to detect infection. A similar approach was used to estimate the necessary accuracy thresholds for any other vital-sign collected by the selected wearable devices.

A power analysis was carried out to estimate the number of subjects needed to reliably detect changes in vital-signs associated with infection. However, because original studies are proposed for which corresponding vital-sign data do not exist, the calculations relied on assumptions based on previous malaria studies [9] and other vital-sign research conducted by the inventors. For the power analysis, the t-test was used, where the null hypothesis was that the means of the two groups (healthy vs. infected) were the same, and the alternative hypothesis was that they were different (i.e., one either greater or less than the other).

Epstein et al. reported that in CHMI studies, at day 10, 60% of subjects have a temperature of >38° C. Assuming a baseline temperature of 37° C. and a standard deviation (SD) of 1.5° C. from in-house testing, our analysis revealed an expected effect size of 0.61, and a minimum sample size of 16 subjects to reliably distinguish (>80% of the time) healthy from malaria-infected subjects at a criterion of P<0.05. Infection studies conducted on non-human primates have shown that during infection, heart rate rises approximately 10%, from 72 bpm to 80 bpm with an SD of 10 bpm. A power analysis based on these assumptions revealed an expected effect size of 0.80, with a minimum sample size of 12 subjects needed to reliably distinguish healthy from malaria-infected subjects at a criterion of P<0.05.

Subjects in the aforementioned CHMI studies were recruited to collect vital-sign measurements throughout the course of the study using the wearable devices. Each subject underwent at least two weeks of baseline lab testing and vital-sign measurements with a wearable device. During a challenge, subjects were exposed to the standard 5-bite CHMI challenge protocol in the WRAIR bridging study, or to 1, 3, and 5 bites in the NMRC dose optimization study. Starting on post-challenge Day 9, all subjects were required to stay in a controlled environment for evaluation up to a maximum of 10 nights. Blood samples were collected daily from post-challenge Day 5 to 18. Parasitemia were detected from the blood samples, using either a blood smear test [4] or qPCR [5]. A standard dose of chloroquine, artemether-lumefantrine, or atovaquone-proguanil were administered to all parasitemic subjects. Subjects were followed for safety and further assessment for three months after the challenge. Subjects used the wearable vital-sign monitoring devices from two weeks prior to challenge, continuously for two weeks following anti-malarial treatment or until the end of the study period.

Study subjects were trained on the use of the wearable devices during a 30-minute session administered two weeks prior to challenge, in which they were given a demonstration on how to use, recharge, and store the devices. Subjects used the wireless wearable devices continuously, from two weeks prior to exposure all the way through to the resolution of the infection. To improve compliance, subjects were given periodic e-mail, text, and phone-call reminders to wear their devices, keep them charged, and transfer data to the paired smartphone, as necessary. During the day of the challenge, the devices were inspected to ensure they are functioning properly, and following the challenge, subjects were given daily reminders to use their devices. During the critical period from Day 9 to 19, when parasitemia is expected to emerge, subjects spent their nights in a pre-determined hotel and underwent daily tests for vital-signs and parasitemia. Their device usage was checked to ensure compliance during this most critical phase of data collection.

2. Develop a Predictive Model for Early Warning Detection of Infection

Continuous vital-sign data from the CHMI study was analyzed to identify physiological changes that are associated with subclinical infection, pre-symptomatic infection, symptomatic infection and then use this information to develop an indicative/predictive model that estimates the likelihood of infection based on real-time vital-sign data in the subjects. Continuous vital-sign data, was collected using wearable devices in everyday, ambulatory conditions, resulted in significant sources of noise and variation in the data. Data pre-processing methods were developed to reliably identify physiological changes reflected in the data, and then AR models trained on the vital-sign data to characterize the persistent physiological changes that predict the likelihood of infection.

Various filtering algorithms (e.g., moving average, median filter) were evaluated to determine the most suitable one that eliminates possible high-frequency noise in the vital-sign data. Vital-sign signals were correlated with subject activity (via 3-axis accelerometer data) to determine scenarios that may produce low-quality data (e.g., a wearable device is not worn or is worn inappropriately). Data collected across all subjects were collected and compared, to identify outlier data that suggested low data quality, and were excluded from subsequent analyses.

Vital-sign data were used to develop and implement data analytical approaches to estimate the likelihood of infection in a time-sensitive manner. This component of the analytics platform employed multivariate AR models [12, 13] trained on vital-sign data collected before malaria infection. Detection algorithms were developed to assess a subject's health status by determining the drift of vital-sign values from model-predicted values. Multiple vital-sign measurements were evaluated and performance of the AR models were examined in detecting infection in univariate and multivariate analysis settings, as well as at multiple time points along the infection timeline, using methods such as the SPRT [14].

The widely applied AR-model formulation [12, 13] was employed to predict vital-sign values reflective of baseline physiological conditions. Given a time series of vital-sign measurements yₙ₋ᵢ at the current time n, with i=0, 1, . . . m−1, a linear combination of these measurements was used in an AR model of order m to estimate the next vital-sign value at time n+1, using the following formulation:

$${\hat{y}}_{n + 1} = {\sum }_{i = 0}^{m - 1} {b}_{i} {y}_{n - i}$$

where bᵢ denotes the AR-model coefficients. A baseline (i.e., pre-infection) vital-sign data was used to “train” the AR models and obtain the bᵢ values, which were estimated by the forward-backward least squares method [12]. In addition to estimating single data points, confidence intervals [7] were estimated/iterated to assess prediction reliability.

A portion of the CHMI data was used to develop AR models for each type of vital-sign. To this end, the model order m, the sampling period S, and the AR-model coefficients were determined and optimized. The optimal m was determined and optimized by computing and analyzing partial autocorrelation functions, and the optimal S by analyzing the magnitude of the power spectra of the vital-sign and choosing S to preserve important features of the signal while minimizing noise.

Malarial infection caused vital-signs to deviate from their baseline values, leading to differences (or residuals) between the observed and AR-predicted values. The challenge was to identify residuals reflective of true infection as early as possible, while minimizing the possibility of triggering a false alert. The best trade-off between timeliness and accuracy was achieved using the SPRT approach [14]. The SPRT used a statistical hypothesis-testing formalism that compared two hypotheses: the null hypothesis (H₀; the subject is not infected) and the alternative hypothesis (H₁; the subject is infected). For a given vital-sign, these hypotheses were represented in terms of the residual X, with mean μ and variance σ². One possible set of values for these hypotheses is given below:

$${H}_{0} \text{:} μ = 0$$

$${H}_{1} \text{:} μ = {μ}_{1}$$

where μ₁ is the mean of the residual when the subject is infected, the value of which can be estimated from the vital-sign data of infected individuals. In the SPRT, likelihood ratio tests of these hypotheses were performed, using the statistic Lₙ at the current time n, defined as follows:

$${L}_{n} = {\sum }_{k = 0}^{K - 1} ln \frac{P r ( {X}_{n - k} \vert {H}_{1} )}{P r ( {X}_{n - k} \vert {H}_{0} )}$$

where the logarithm of the ratio, of the probability density of the observed vital-sign residual Xn-k at time n−k under the alternative hypothesis to that under the null hypothesis, is summed over all time points within a selected time window of length K. The decision criteria for the statistic were as follows: Accept H₀ if Lₙ<B; Accept H₁ if Lₙ>A; otherwise make no decision and proceed in time, where the constants A and B (A>B) determine the false positive and false negative rates, where μ₁ is the mean of the residual when the subject is infected, the value of which can be estimated from the vital-sign data of infected subjects. In the SPRT, likelihood ratio tests of these hypotheses were performed, using the statistic Lₙ at the current time n, defined as follows:

$${L}_{n} = {\sum }_{k = 0}^{K - 1} ln \frac{P r ( {X}_{n - k} \vert {H}_{1} )}{P r ( {X}_{n - k} \vert {H}_{0} )}$$

where the logarithm of the ratio, of the probability density of the observed vital-sign residual Xn-k at time n−k under the alternative hypothesis to that under the null hypothesis, is summed over all time points within a selected time window of length K. The decision criteria for the statistic are as follows: Accept H₀ if Lₙ<B; Accept H₁ if Lₙ>A; otherwise make no decision and proceed in time, where the constants A and B (A>B) determine the false positive and false negative rates, respectively. These SPRT algorithms were applied to each physiological vital-sign to detect statistically significant deviations from normal baseline values.

Performance of the predictions generated by the SPRT algorithms described above were evaluated by calculating sensitivity and specificity. These performance measures were defined with respect to the true infection status, which is “healthy” for time points before exposure and “infected” for those after exposure. Under a given choice of parameters A and B, which control the diagnostic decision in the SPRT, time segments for each vital-sign time-series data were obtained for which the diagnostic outcome is either “healthy” (H₀ accepted) or “infected” (H₁ accepted for the full test). An initial alert was generated when the diagnostic outcome changes from “healthy” to “infected.” With CHMI data, sensitivity and specificity was then defined as the fraction of time points after exposure with the “infected” diagnosis and that before exposure with the ‘healthy’ diagnosis, respectively. Finally, combinations of SPRT and AR-model parameters were identified (sampling period S; prediction window; time-window length K; and parameters A, B, and bᵢ) that led to the best balance between the accuracy (sensitivity and specificity) and timeliness of the decision. The accuracy of the detection model was evaluated as a function of both time-to-illness and time-from-exposure.

3. Blindly Validate Infection-Detection Model in a Second Controlled Human Infection Study

The predictive model for detecting pre-symptomatic, subclinical infection was blindly assessed and then validated in a second, independent CHMI study. A wide range of vital-sign data as possible was collected and physiological measures predictive of infection were identified. Vital-sign data only for those physiological measures found to be predictive for a specific infection were collected. Predictive models were validated through internal cross-validation, using subsets of subjects used to train the model in the same study. However, using a second, independent study ensured that the predictive accuracy is robust to differences between studies and subjects. This also allowed for blind validation, in which predictions were performed before the true status of the subjects was disclosed.

Once a detection model for predicting the likelihood of malaria infection in subjects in real time from vital-sign data was developed and tested, a second CHMI study was performed, in which balanced numbers of subjects were infected and not infected. During this study, as before, subjects were asked to use the wearable devices continuously throughout the course of the study. Once data collection was completed, the inventors, who remained blind to the infection status of the subjects, applied the detection model to the wearable-derived vital-sign data collected from each subject to determine whether that subject had a malaria infection and, if so, how quickly, post-exposure, the model made a determination.

Once unblinded study results were obtained, the accuracy (sensitivity and specificity) and timeliness of the indication/prediction against clinical data were evaluated. Accuracy was measured in terms of the model's positive predictive value and specificity was determined and assessed as a function of time-from-exposure and time-to-illness. This allowed an independent evaluation of the model's ability to generate an alert for possible infection in terms of its accuracy and potential for generating false positives. Furthermore, because the model was parameterized using the initial data collected in a separate CHMI study, this independent validation assessed the degree to which the model was generally applicable to multiple CHMI studies and potentially a range of infection conditions. Finally, by using data from two independent CHMI studies, simpler SPRT models were assessed and compared, built purely from a separate data set of uninfected subjects, that indicated/predicted infection as well as models that used subject-matched baseline and infection data—i.e., whether a group-level model was sufficient to predict infection, or individualized models using subject-matched baseline data were necessary.

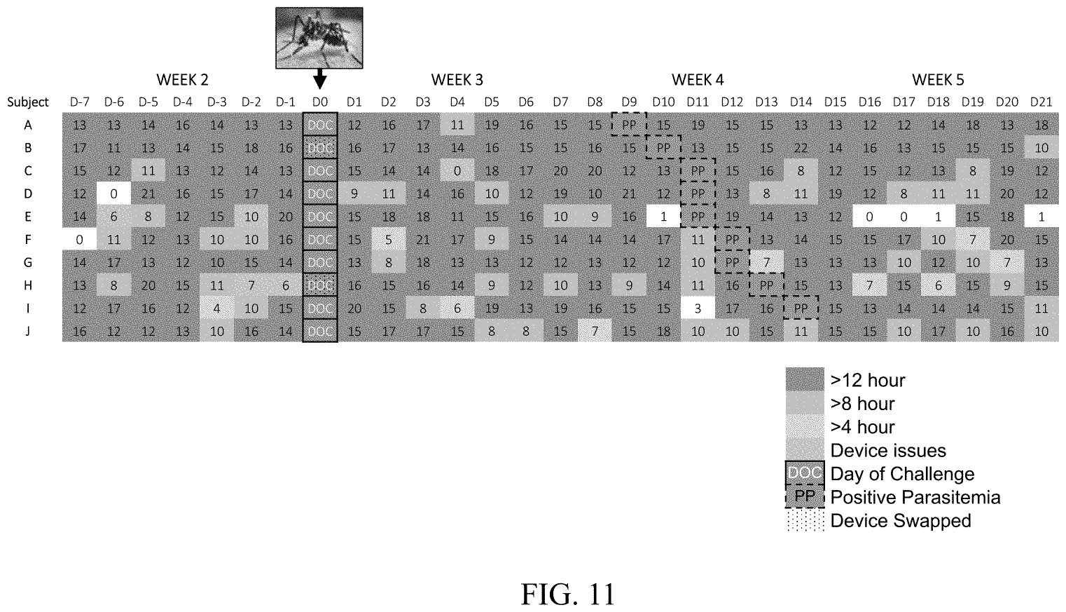

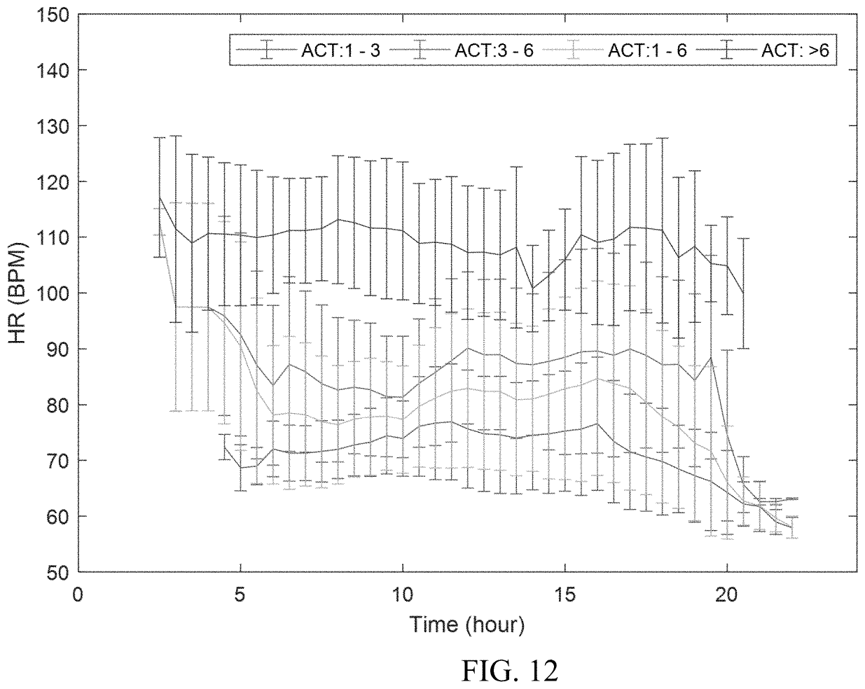

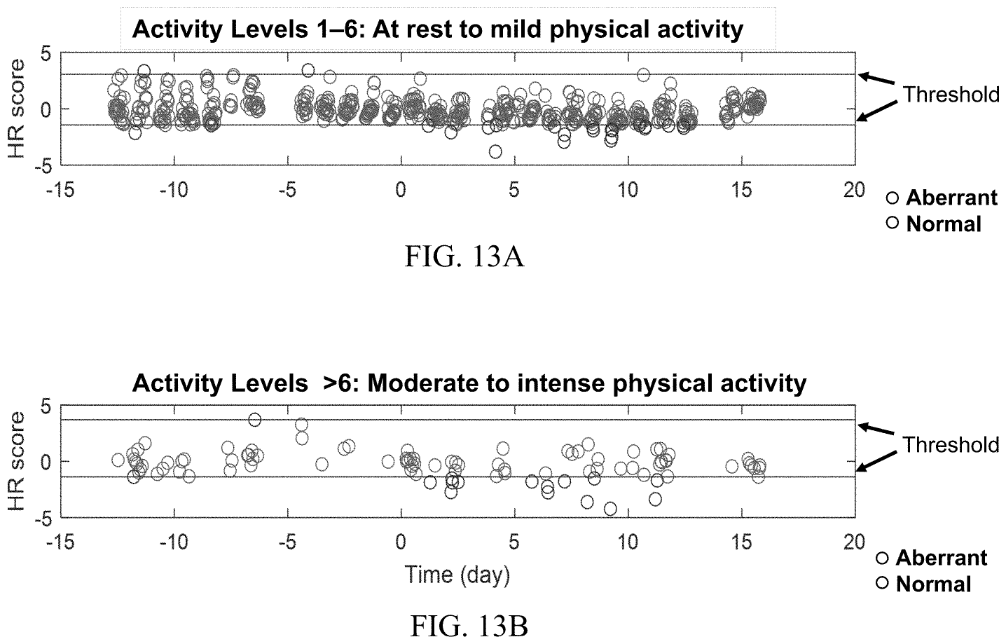

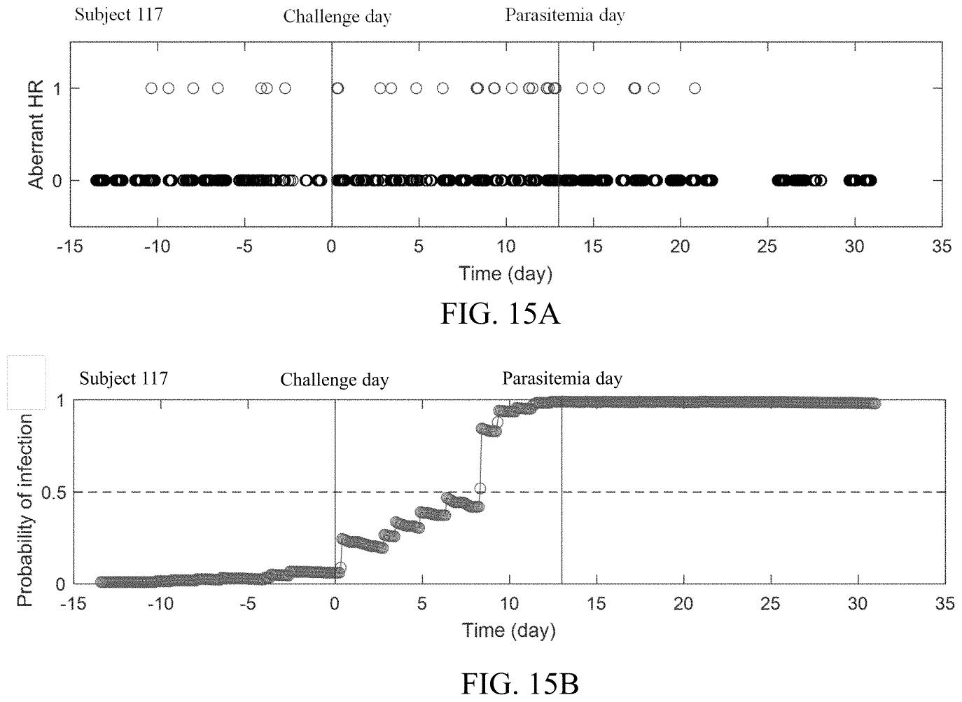

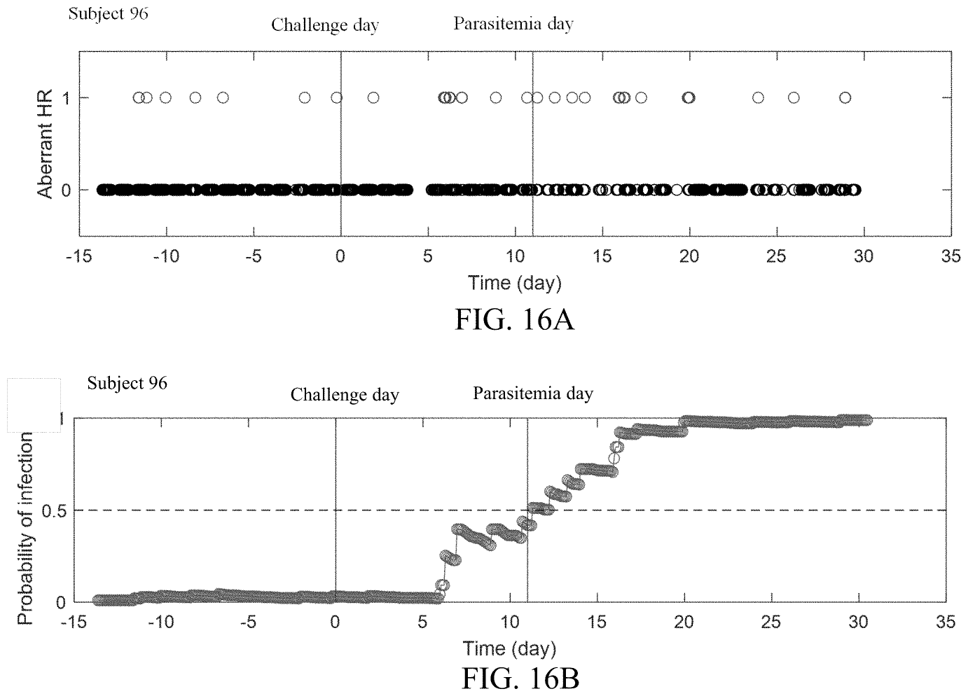

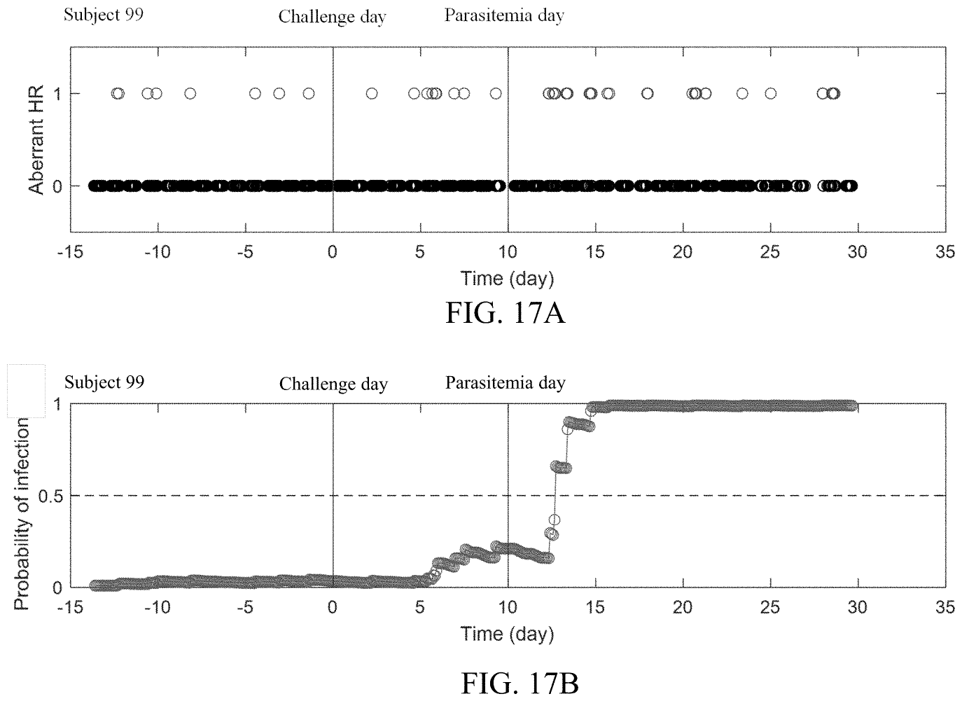

FIGS. 11-20E illustrate some of the underlying data from the CHMI study discussed above. FIG. 11 illustrates the level of data collection during the CHMI study from Week 2 to Week 5. There was a 72% compliance for 12+ hour/day and a 92% compliance at 8+ hour/day. FIG. 12 illustrates how the level of activity (ACT) and the heart rate (BPM) will vary during a day. FIGS. 13A and 13B provide examples of how vital-sign thresholds might be overlaid with heart rate scores to determine which heart rate scores were normal and which were aberrant using two different ranges of activity level.

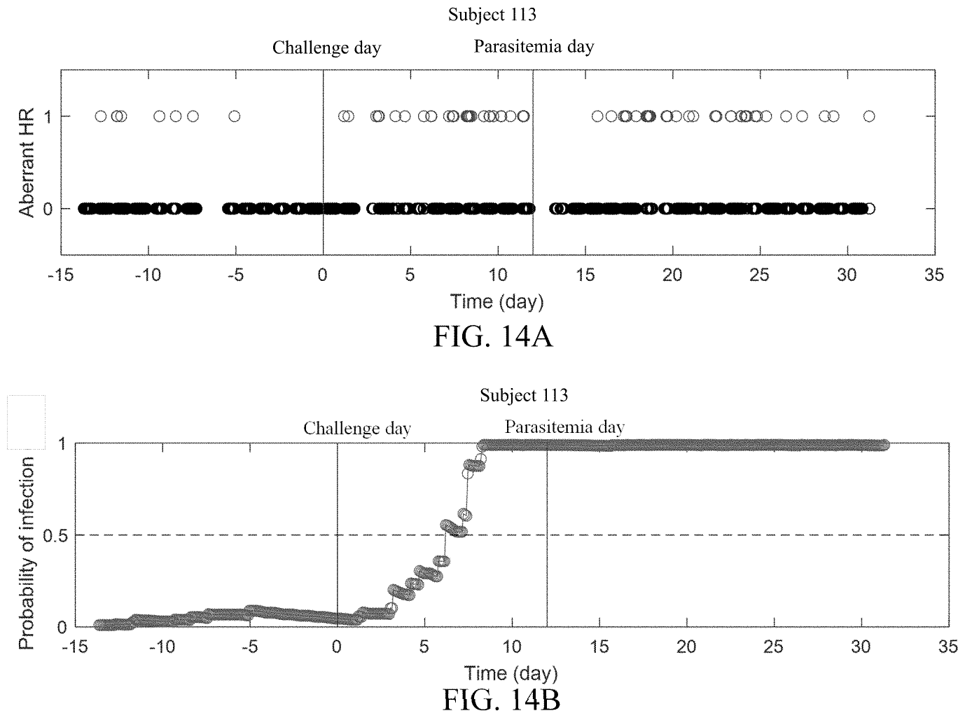

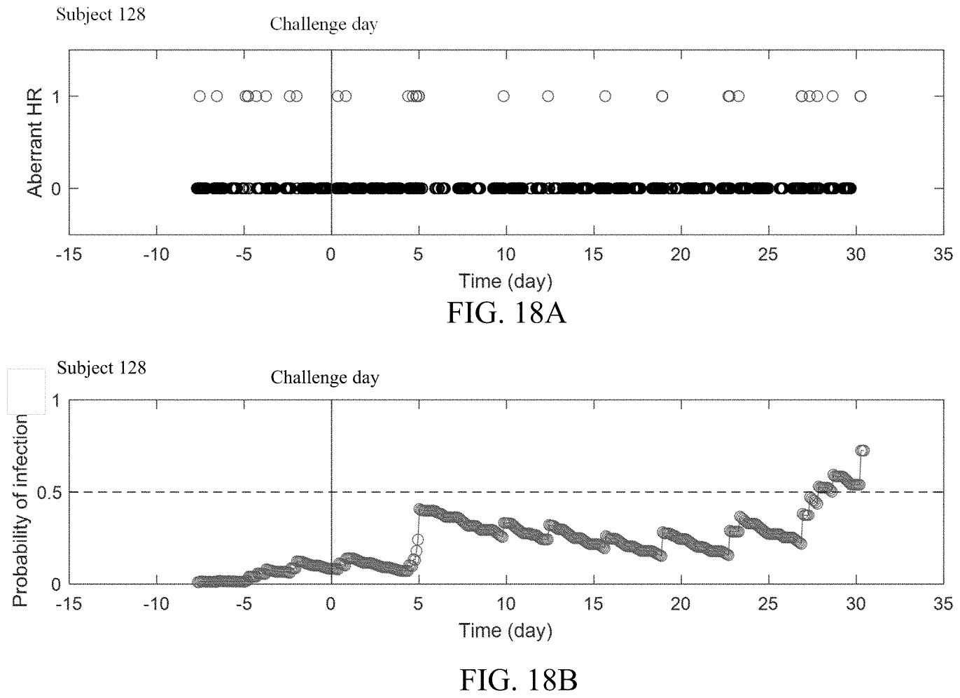

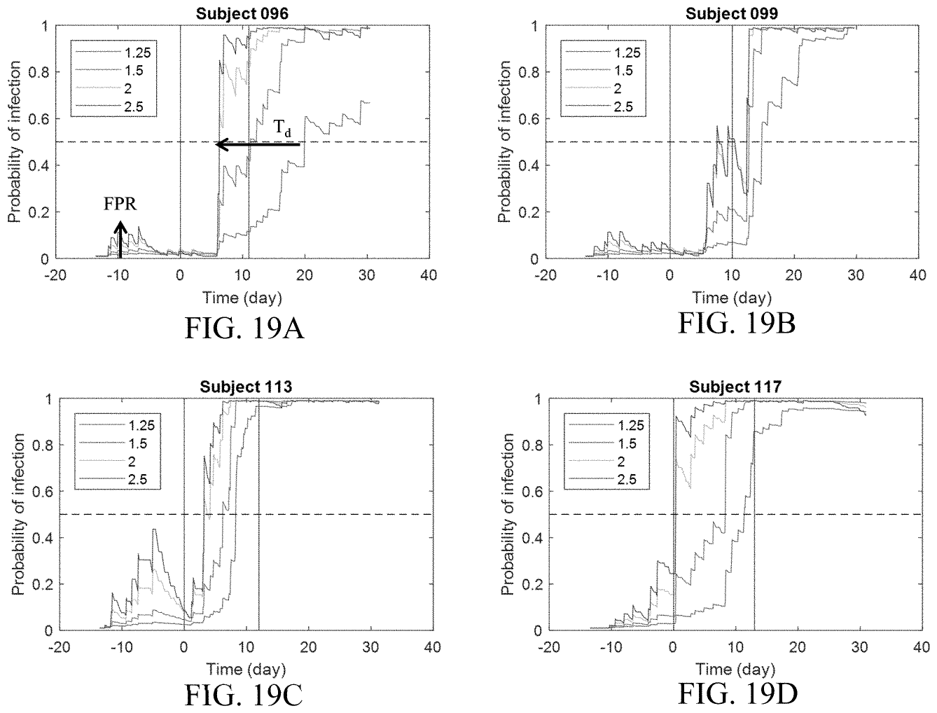

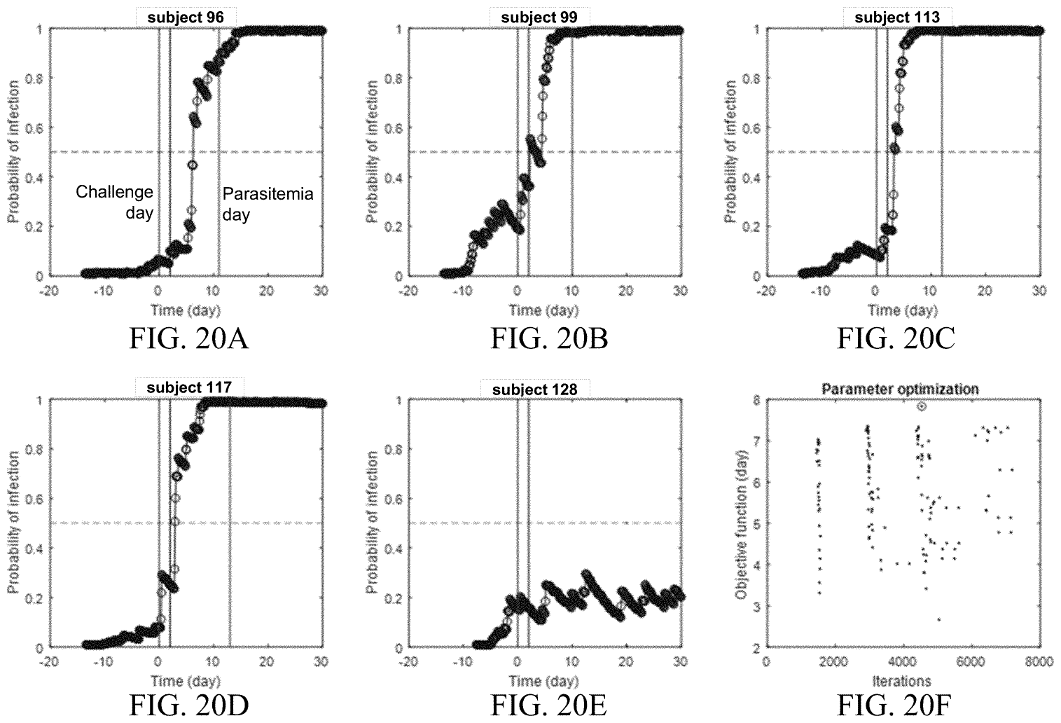

FIGS. 14A-18B illustrate data generated from five different study subjects illustrating whether an aberrant heart rate was detected (0 equates to normal and 1 equates to aberrant) and how the infection probability (using the probability model) changed over the time of the CHMI study. For Subject 128 whose data is illustrated in FIGS. 18A and 18B, the subject did not receive a positive test for a parasitemia despite having been exposed to infected mosquitos. FIGS. 19A-19D illustrate true positive rate factors in the CHMI study for four of the subjects using four different levels of sensitivity for the probability of infection as compared to the exposure date (Day 0) and the detection date as represented by the vertical line to the right of the exposure date. FIGS. 20A-20E illustrate optimized probability models for the timeliness of the probability of infection for the five subjects in the CHMI study whose data is depicted in FIGS. 14A-19D. FIG. 20F illustrates parameter optimization based on study data.

This study data was analyzed using one method embodiment in conjunction with particular hardware, as discussed later, to collect the vital-sign data for the baseline phase and the detection phase.

4. Implement Analytical Tools in a Real-Time Detection System to be Made Available as an App

Analytical tools were developed and translated to indicating/predicting subclinical, pre-symptomatic and symptomatic infection into a prototype real-time detection system that integrated hardware, such as commercially available wearable devices and smartphone, with analytical software that carried out physiological monitoring and implemented the infection prediction algorithm. A commercial prototype likely will have an interface that informs/warns/alerts users about their health status. Furthermore, because the end user may need to remotely access the tools, these tools can be optimized and/or customized so that they are available as a downloadable app.

A predictive model that uses vital-sign data collected from commercially available wearable devices was developed and validated to accurately estimate the likelihood of infection in subjects. Analytical tools were then translated to making real-time predictions into in an integrated commercial prototype system. First, the predictive model was adapted to make predictions fast enough to provide the user with a health status assessment in real time. Second, analytical software was developed that can be loaded onto an Android smartphone, receive continuous vital-sign data from the corresponding wearable devices, and run the prediction algorithm to make real-time predictions, all on the smartphone itself—without the use of any Internet or network resources. In theory, the prototype system should be modular/mobile, so as to be adaptable to new wearable devices as they become available.

Analytical tools were adapted, modified and customized as needed to be usable as apps by third-party users. In addition, a graphical user interface was developed to facilitate usability.

End users and direct beneficiaries of the proposed technology are civilians and deployed service members. By developing a continuous vital-sign monitoring system using commercially available wearable devices, the proposed warning system will passively and unobtrusively monitor individuals, including civilian and military, bypassing the need for active end-user effort and minimizing obstruction to the user. Importantly, the system extends the state-of-the-art in wearables by transforming data (i.e., physiological measurements) into actionable information (i.e., corroborating evidence of infection exposure).

F. Example Implementation

The model to analyze heart-rate and actigraphy data and provide a real-time estimate of the probability of infection includes two steps: 1) detection of aberrant heart-rate (AHR) patterns and 2) real-time estimation of probability of infection through the accumulation of elevated AHR frequencies over time.

1. Data Pre-Processing

The raw heart-rate data were collected at a sampling rate of 1 Hz, and then averaged in non-overlapping 5-second windows. A moving average was then taken using a 15-minute window. The activity data were collected by a 3-axis accelerometer at 25 Hz, and averaged in a non-overlapping 5-second window along each axis. The 5-second mean acceleration along the three axes was converted into a magnitude of acceleration vector. This value was then used to calculate a metabolic equivalent level (MET level) using an in-house algorithm [23], which algorithm is hereby incorporated by reference, based on the previously referenced Sasaki algorithm. The in-house algorithm showed high correlation with the original version, but was developed based on 125 hours of activity data collected in-house that was carefully annotated by activity (e.g., “sleeping,” “office work,” “walking,” and “exercising”). A moving average of the MET levels was then taken with a 15-minute window, resulting in pre-processed heart rates at 5-second intervals paired with MET levels at the same time intervals.

2. AHR Detection

In order to identify AHRs, the model first generated an individualized baseline distribution of “healthy” (non-infection) heart rates at different times of day and levels of activity. The baseline heart rate data were obtained from the pre-challenge time period for CHMI subjects, and from the first two weeks of data collection for the control subjects. The activity levels were obtained by converting the raw acceleration data into MET values [23] and then mapping these values into three activity levels: “resting,” “low,” and “moderate.” To define the MET cutoff values associated with each activity level, an activity-annotated dataset was used, where the acceleration data and associated activity annotation were previously collected using the Samsung Gear S3 smartwatch. Based on these activity-annotated data, the MET values were defined as follows: MET values of 0.50 to 0.75, which included annotated activities such as “sleep,” as an activity level of resting; MET values of 0.75 to 1.00, which included annotated activities such as “office work,” as an activity level of low; and MET values of 1.00 to 1.25, which included annotated activities such as “walking,” as an activity level of moderate. Table 3 below provides additional information. Higher activity levels were excluded because too little data were collected for reliable baseline estimates.

TABLE 3
Metabolic equivalent (MET) level cutoffs for each of the three
activity levels based on activity-annotated data.

| | Example Activity | Number of | | MET Cutoffs |
|---|---|---|---|---|
| Activity Level | Annotation | data points | Lower | Upper |
| Resting | ″Sleep″ | 38,836 | 0.50 | 0.75 |
| Low | ″Office Work″ | 11,764 | 0.75 | 1.00 |
| Moderate | ″Walking″ | 38,820 | 1.00 | 1.25 |

For each subject, baseline distributions at different times of day (tₖ) about a window of time (±w), at each of three activity levels (aⱼ) were determined. For a given query period, the average heart rate over a 30-minute window [HR(tₖ, aⱼ)] was calculated from the pre-processed heart-rate data annotated by the corresponding time of day and activity level during that time window. This average heart rate was then converted to a heart rate score for a time window tₖ±w and activity level aⱼ, based on the mean μ and standard deviation a of the baseline distributions as described in the following equation:

$$HR Score = \frac{H R ( {t}_{k} , {a}_{j} ) - μ ( {t}_{k} \pm w , {a}_{j} )}{σ ( {t}_{k} \pm w , {a}_{j} )}$$

A minimum and maximum cutoff for the heart rate score was defined, outside of which a heart rate was considered to be an AHR. These cutoffs were based on the upper and lower bounds corresponding to 95^{th} percentile limits of the baseline distribution and were calculated by fitting a Weibull distribution to the baseline distributions for each subject, at each activity level. Parameterization, in terms of finding appropriate values for the window of time (w), heart-rate average window (30 minutes), and threshold cutoffs (95^{th} percentile), was determined manually with the goal of obtaining heart rate scores and AHR classifications that were robust to week-to-week variations in baseline data, while being sensitive to clusters of abnormally elevated or decreased heart rates. These analyses were carried out using both simulated and real-world wearable-collected data. One important advantage to using an activity-level specific, percentile-based approach to detecting AHRs is that it accounts for the greater noise in heart rate data at higher activity levels by comparing all heart rate data to baseline data collected at that same activity level. Thus, as long as the noise with respect to activity level is comparable between the baseline and detection periods, increased noise at higher activity levels will not result in higher AHR frequencies.

3. Real-Time Estimate of Infection Probability

This particular approach relies on a Bayesian-based model for estimating real-time infection probability by accumulating evidence of AHRs over time. The real-time probability of infection at time t (Pₜ) about a monitoring time window of 30 minutes is a function of the number of aberrant (n) and normal (k) heart rates observed in the time window around time t, along with the prior probability of infection at the previous time window (Pₜ₋₁), as described in the equation below. Additional fixed parameters include the probability of observing AHRs in healthy (Pₕ) and infected (Pᵢ) individuals. There is also a “forgetting” factor (α), with α<1.0, which causes the contributions of prior probabilities to the current probability to decay with time, creating a recency bias to Pₜ. The equation is based on Bayes theorem and estimates the posterior probability of infection Pₜ, at time t, based on a prior probability of infection, the likelihood of observing normal and aberrant heart rates in healthy and infected individuals, and the new evidence—the number of newly observed normal and aberrant heart rates at time t. In the extreme case where no heart rates are observed, the posterior probability is equivalent to a multiplied by the prior probability, leading to a slight decrease in the prior probability of infection reflecting the lack of recent data. In cases where no normal or aberrant heart rates are observed, Pₜ will increase or decrease, respectively, based on the parameters Pₕ and Pᵢ and the prior probability.

$${P}_{t} = \frac{1}{1 + {( \frac{{P}_{h}}{{P}_{i}} )}^{n} {( \frac{1 - {P}_{h}}{1 - {P}_{i}} )}^{k} ( \frac{1}{α {P}_{t - 1}} - 1 )}$$

Values for fixed parameters Pₕ and Pᵢ were set based on the goal of obtaining the maximum separation in peak infection probability between wearable data obtained from CHMI subjects (during the incubation phase) and control subjects. A supervised machine-learning approach to parameter fitting was not used because of the small sample size of the study and the high risk of over-fitting. Instead, the parameters were manually set based on key desired behaviors of the model: 1) Pᵢ>Pₕ, based on the assumption that infection leads to an increase in AHR frequency; 2) Pₕ=0.05, reflecting the AHR thresholds set about the limits of the 95^{th} percentile of the baseline (healthy) distribution; and 3) AHRs observed more than 5 days prior should be ignored based on the assumption that early stages of infection rarely exceed this time span. The a parameter was adjusted so that the contribution of AHRs observed 5 days or earlier becomes zero. Since Pᵢ is not known, its value was adjusted, as a ratio of Pₕ, and observed its effect on Pₜ; this effectively adjusted the sensitivity of the prediction model to the presence of AHRs and is the single-most important parameter in this particular model.

4. AHR Detection in Study Data

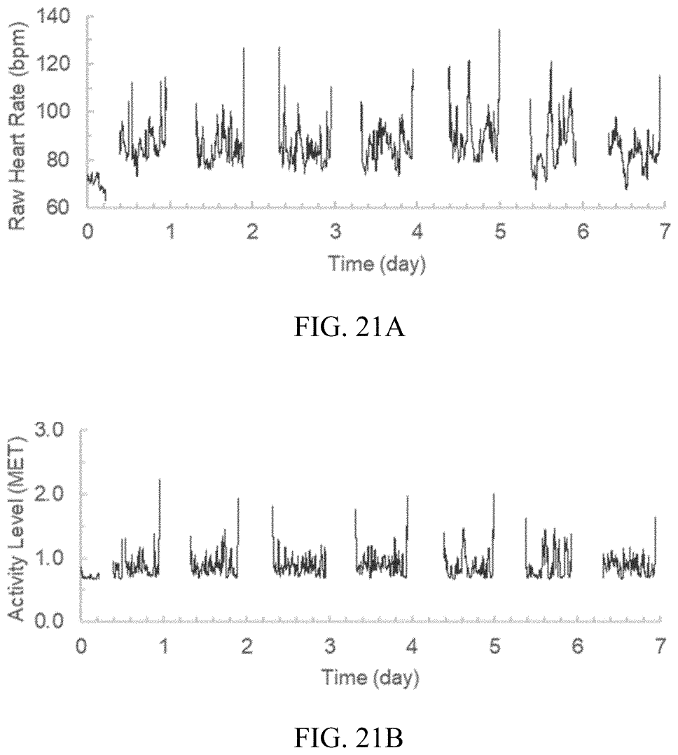

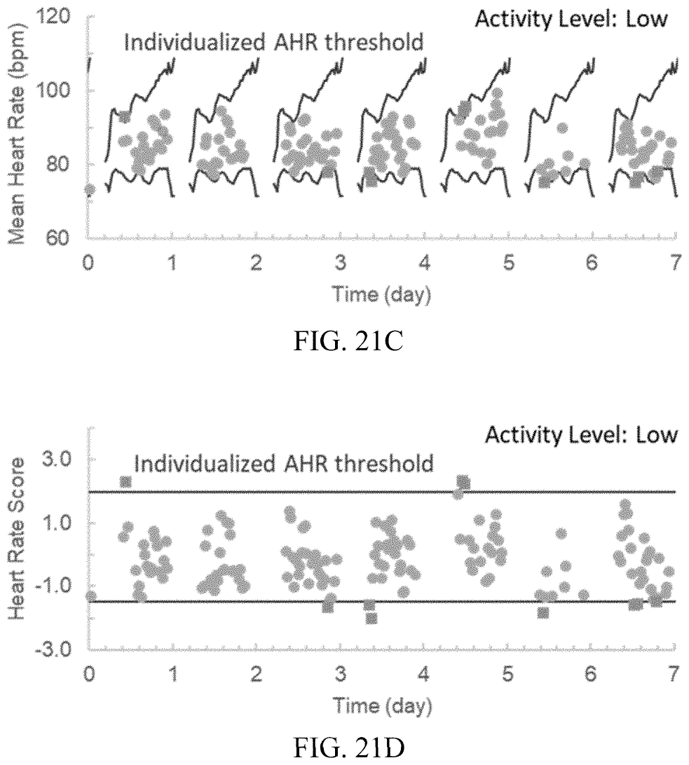

The raw heart-rate data and the actigraphy data were combined to generate a heart rate score that took into account circadian rhythm and activity, and then implemented an AHR classification scheme whereby any heart rate score outside the thresholds defined by the 95^{th} percentile limits of the corresponding baseline distributions was classified as “aberrant” and all others were classified as “normal.” FIGS. 21A-21D show representative heart-rate (FIG. 21A) and actigraphy data (FIG. 21B) collected from a single subject over a 7-day period that were converted into average heart rates and finally into heart rate scores across all times of day, for each activity level, with those of low activity level shown in FIGS. 21C and 21D, respectively.

The overall frequency of AHR (relative to total number of heart-rate data points) for both the baseline and the post-baseline monitoring period is shown in Table 4 for CHMI and control subjects. Because the baseline data were used to define the 95^{th} percentile heart rate score thresholds that define AHRs, it is expected that the AHR frequency during the baseline period should be approximately 5%.

TABLE 4
Overall frequency of AHRs for the baseline and post-baseline monitoring periods

| | Baseline | | | Monitoring Period | | |
|---|---|---|---|---|---|---|
| Subject | #AHRs | #NHRs | % AHR | #AHRs | #NHRs | % AHR |

CHMI

| | | | | | | |
|---|---|---|---|---|---|---|
| 96 | 19 | 363 | 5.0 | 48 | 537 | 8.2 |
| 98 | 13 | 166 | 7.3 | 75 | 399 | 15.8 |
| 99 | 14 | 245 | 5.4 | 60 | 497 | 10.8 |
| 103 | 5 | 141 | 3.4 | 60 | 325 | 15.6 |
| 109 | 23 | 410 | 5.3 | 70 | 900 | 7.2 |
| 111 | 25 | 488 | 4.9 | 75 | 702 | 9.7 |
| 113 | 22 | 306 | 6.7 | 138 | 657 | 17.4 |
| 117 | 23 | 358 | 6.0 | 112 | 640 | 14.9 |
| 118 | 27 | 402 | 6.3 | 57 | 501 | 10.2 |
| 128 | 12 | 190 | 5.9 | 52 | 762 | 6.4 |
| Average | 18 | 307 | 5.6 | 75 | 592 | 11.6 |

Control

| | | | | | | |
|---|---|---|---|---|---|---|
| 1 | 20 | 407 | 4.7 | 24 | 457 | 5.0 |
| 2 | 8 | 271 | 2.9 | 33 | 244 | 11.9 |
| 4 | 15 | 385 | 3.8 | 23 | 256 | 8.2 |
| 5 | 11 | 189 | 5.5 | 10 | 230 | 4.2 |
| 6 | 31 | 433 | 6.7 | 55 | 443 | 11.0 |
| 7 | 15 | 307 | 4.7 | 14 | 244 | 5.4 |
| 8 | 26 | 411 | 5.9 | 54 | 493 | 9.9 |
| 9 | 20 | 367 | 5.2 | 27 | 287 | 8.6 |
| Average | 18 | 346 | 4.9 | 30 | 332 | 8.0 |

AHRs = abnormal heart rates;
NHRs = normal heart rates

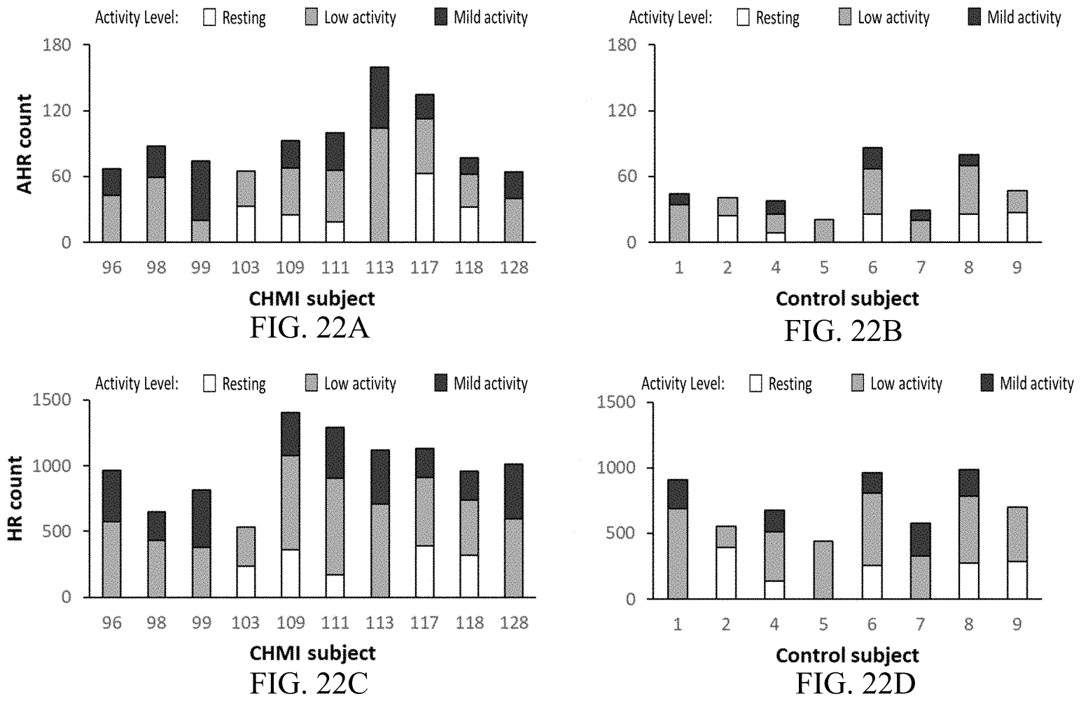

Overall, the average AHR frequency found in the baseline period was 5.6% for CHMI subjects and 4.9% for control subjects. For CHMI subjects during the post-challenge monitoring period, the average AHR frequency rose about 2-fold to 11.6%, while in the control subjects, the average AHR frequency during the post-baseline monitoring period was 8.0%. Both CHMI and control subjects showed an increase in AHR frequency from baseline (P<0.01 and P<0.05, respectively). However, CHMI subjects showed a significantly larger increase in AHR frequency than control subjects (P<0.05), suggesting that increased AHR frequency may be indicative of infection. Furthermore, as illustrated in FIGS. 22A-22D, AHRs were observed at all three activity levels, in both CHMI and control subjects, respectively, roughly corresponding to the amount of wearable data collected at each level, with the most AHRs detected at the “low” activity level. For some subjects, no AHRs were observed at the “resting” activity level because there were insufficient wearable data collected at this activity level for those subjects.

5. Estimating Probability of Infection in the Study Data

A Bayesian-based model was developed for estimating the probability of infection at a given time point (Pₜ) from AHR frequency data. The model includes three main parameters: 1) Pₕ, the probability of observing an AHR in a healthy (non-infected) individual; 2) Pᵢ, the probability of observing an AHR in an infected individual; and 3) α, a decay factor that biases Pₜ to more recent observations.

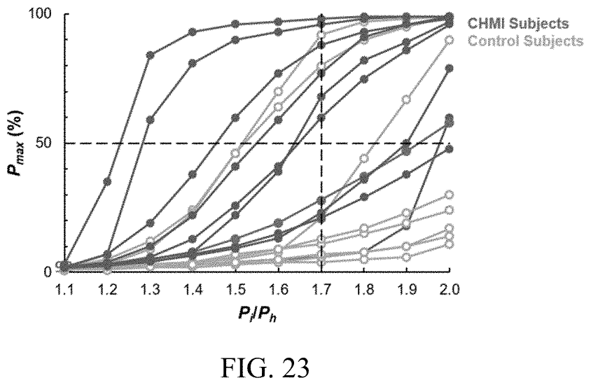

FIG. 23 shows the effect of adjusting Pᵢ, as a ratio of Pₕ, on the maximum observed Pₜ during the incubation period (CHMI subjects) and total monitoring period (control subjects). The inventors found that there was separation in the maximum observed Pₜ between CHMI and control subjects across a range of values for Pᵢ/Pₕ from 1.6 to 1.9. For most CHMI subjects (6 of 9 that achieved parasitemia), their maximum Pₜ exceeded 40% in this range of Pᵢ/Pₕ. Based on the observed separation, a Pᵢ of 1.7*Pₕ was selected in order to calculate Pₜ, and a Pₜ threshold of 50% above which a subject would be classified as “infected” in the model. With Pᵢ/Pₕ at 1.7, the “forgetting” factor α was adjusted to be 0.988 based on simulated heart-rate data.

6. Infection-Detection Model Performance

Table 5 summarizes the infection-detection performance in CHMI and control subjects. Infection was detected in seven of nine CHMI subjects that developed parasitemia; in six of nine subjects it was detected prior to the detection of parasitemia by blood smear, with an average T_{d} of 6.4 days. Four CHMI subjects reported moderate or severe CHMI-related symptoms within 2 weeks post-challenge; in all four cases infection was detected (Pₜ>50%), with an average Tₛ of 3.3 days. In control subjects, Pₜ exceeded the 50% threshold in two of eight cases, over a combined monitoring of 32 weeks. Overall, the sensitivity for infection detection was 78% (7 of 9), the sensitivity for infection detection prior to parasitemia was 67% (6 of 9), and the false-positive rate based on control subjects was 6% per week, for an overall specificity of 75%.

TABLE 5
Infection detection performance in CHMI and control subjects

| | | | Tₛ, (days) | Control | |
|---|---|---|---|---|---|
| CHMI Subject | Pₘₐₓ(%) | T_{d}(days) | [Severity] | Subject | Pₘₐₓ(%) |
| 117 | 99 | 10.0 | | 004 | 4 |
| 113 | 96 | 4.6 | 5.6 [Moderate] | 007 | 5 |
| 111 | 88 | 4.4 | | 001 | 11 |
| 103 | 77 | 8.1 | | 005 | 13 |
| 98 | 68 | 3.5 | 4.5 [Severe] | 009 | 18 |
| 118 | 83 | 7.5 | 5.5 [Moderate] | 002 | 35 |
| 96 | 29 | −1.3 | −2.3 [Severe] | 008 | 76 |
| 109 | 23 | | | 006 | 96 |
| 99 | 6 | | | | |
| 128* | 17 | | | | |

*CHMI subject 128 was challenged but did not achieve parasitemia.

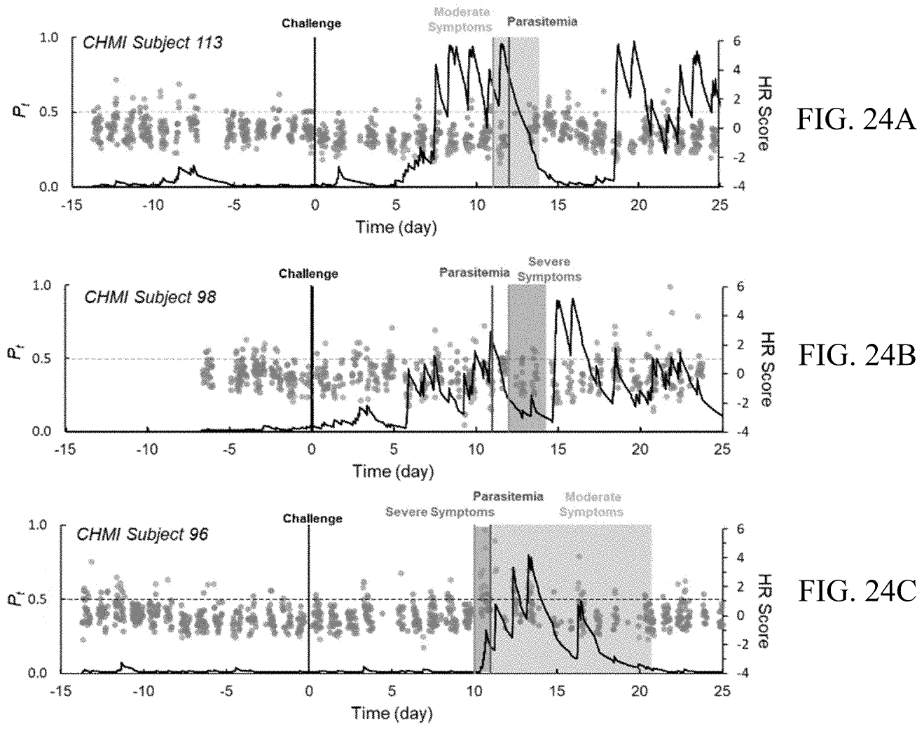

Subject 113 (FIG. 24A) serves as a representative example for strong performance by the model, as measured by Pₘₐₓ. This subject experienced moderate symptoms (headache) on Day 11 and reached blood-stage parasitemia by Day 12. Pₜ for this subject exceeded the 50% threshold at Day 7, 4.6 days prior to blood-stage parasitemia, reaching a Pₘₐₓ of 96%. Subject 98 (FIG. 24B) represented the median performance of the model as measured by Pₘₐₓ. This subject had blood-stage parasitemia by Day 11 and experienced severe symptoms (fatigue) and moderate symptoms (malaise and myalgia) on Day 12. Pₜ for this subject reached the 50% threshold at Day 7, 3.5 days prior to parasitemia. Finally, subject 96 (FIG. 23C) represents a poor prediction by the model. This subject presented blood-stage parasitemia on Day 11 and severe symptom (fever) onset on Day 10. The model did not show Pₜ greater than 50% during the incubation period, but did reach that threshold on Day 12, 1.3 days after blood-stage parasitemia was detected by blood smear.

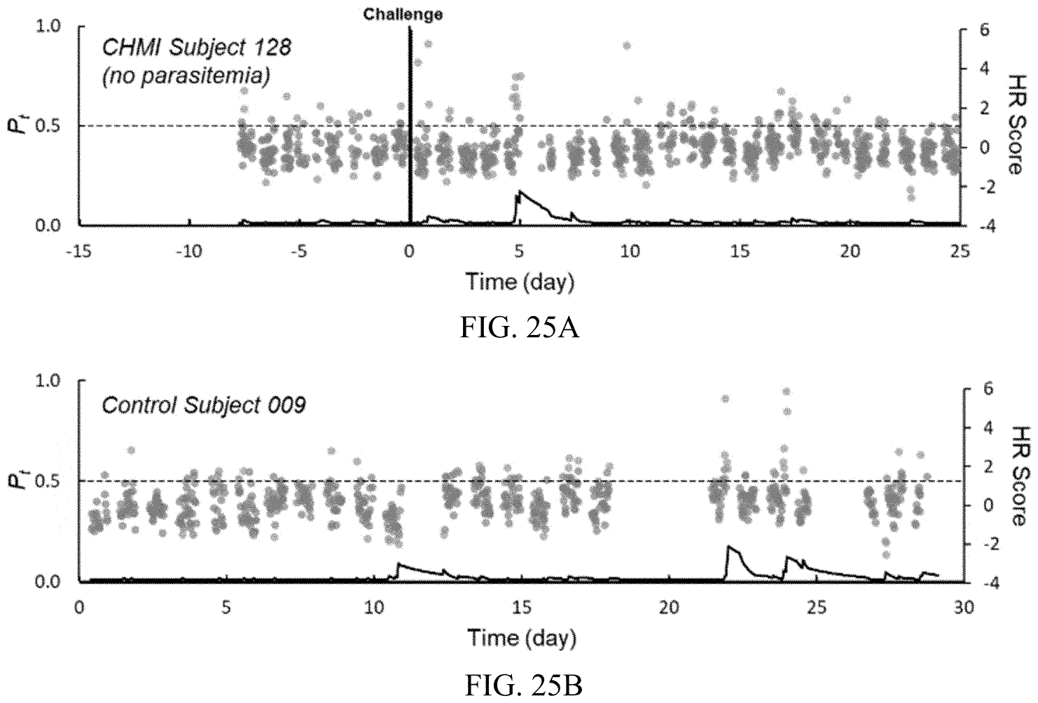

FIGS. 25A and 25B show two examples of model infection-detection performance in subjects that did not have infection. CHMI subject 128 (FIG. 25A) underwent challenge but did not develop parasitemia. Consistent with the blood-based diagnostic result and the lack of malaria-related symptoms, the model also did not report a Pₜ value exceeding the 50% threshold at any point during the post-challenge monitoring period. Likewise, in control subject 009 (FIG. 25B), the median subject in the control group in terms of prediction performance as assessed by Pₘₐₓ, the Pₜ reported by the model was close to zero for almost the entire duration of the monitoring period.

G. SARS-CoV-2 Proposed Study

A study has been proposed to gather data regarding the effectiveness of the invention in predicting infections caused by SARS-CoV-2.

The novel coronavirus has impacted and continues to impact the U.S. economy and military readiness. The ability to intervene early by identifying pre-symptomatic SARS-CoV-2-infected individuals will help decrease the number of contacts and the spread of the virus in the population, especially for those in close working and living conditions. To date, there are no commercially available wearable technologies that have demonstrated the ability to detect SARS-CoV-2 infections in asymptomatic patients.

The proposed effort combines elements of traditional physiological modeling with hardware suitable for field environments to carry out real-time physiological monitoring of subjects, under every day, ambulatory conditions. The vital-sign data collected in this study will be critical for assessment of the false positive rate (FPR) of the detection algorithm for identifying early additional physiological markers of infection and determining the feasibility of developing real-time predictive models that can be incorporated in an early infection warning platform to allow for rapid situational awareness and time-sensitive deployment of countermeasures, such as evacuation, quarantine, and treatment.

A continuous vital-sign monitoring system using commercially available wearable devices, the proposed warning system will passively and unobtrusively monitor individuals, bypassing the need for active end-user effort and minimizing obstruction to the individuals' activity. Importantly, the system extends the state-of-the-art in wearables by transforming data (i.e., physiological measurements) into actionable information (i.e., corroborating evidence of infection exposure). The objective of this study is to collect continuous vital-sign data from an adult population using the Samsung Galaxy Active 2 smartwatch, or equivalent, along with a twice-daily self-reported health status, to assess the invention. The specific aims of this research are to: 1) assess the performance of the invention during near real-time, automatic operation; and 2) assess the ability of the invention platform to identify infected or ill subjects before the onset of symptoms. It is proposed to collect continuous vital-sign data (>12 hours/day) for a 26-week observation period in adult subjects, along with twice daily self-reported health status updates.

This will be an observational study in which vital-sign data will be collected, including heart rate, skin temperature, and 3-axis body acceleration, from subjects as well as a twice-daily self-reported wellness questionnaire. Besides wearing the smartwatch for at least 12 hours per day, subjects will be instructed to continue their regular everyday activities.

The study is planning to recruit 40 adult volunteers (men and women) between the ages of 18 and 60 years and self-reportedly healthy at the time of recruitment. They must be able to speak and read English, give informed consent, and use a smartphone and smartwatch. The exclusion criteria are that subjects will be excluded if they cannot speak and read English or cannot use a smartphone or a smartwatch. Volunteers will be interviewed in accordance with the approved protocol.

Training on device usage will be provided at the beginning of the study and written instructions will be provided to the study volunteers after consent is obtained. The handout will include step-by-step instructions, examples of device usage, and screenshots. The on-site principal investigator will be in contact with volunteers to remind them to follow the study instructions and assist with any questions.

Each volunteer will be issued a Samsung Galaxy Active 2 smartwatch and a Samsung Galaxy S20 smartphone installed with the Physiological Data Recorder (PDR) app, which they will use for the duration of the study to collect vital-sign and self-reported wellness data. The volunteers will be responsible for charging the smartwatch every day, and wearing the smartwatch for at least 12 hours/day on a regular schedule. The volunteers will be advised to leave the smartphone at home, docked to a charger, at all times, unless otherwise instructed. Twice each day, the volunteers will be requested to complete a wellness status questionnaire on the smartphone. The probability algorithm will run on the smartphone to identify aberrant heart-rate values and estimate the likelihood of infection. However, although the volunteers will be able to view their collected (raw) heart-rate values, they will be blinded to heart-rate values (or scores) that are classified as aberrant as well as the estimated infection probability. Approximately once each month, volunteers will be asked to bring their smartphone to BHSAI to have their data downloaded to a BHSAI computer. Although it would be possible to use a network (e.g., the Internet and/or a cellular network) to transfer the data with the appropriate precautions being taken to transmit the health data.

A heart rate (HR) will be classified as normal or aberrant as follows. First, using the baseline data, a normalized heart rate score S is computed with S=(HR−μ)/σ, where μ and σ denote the mean and standard deviation, respectively, of raw baseline heart rates. Next, two thresholds are selected one above and one below S. Then, when a new raw heart rate value falls within the two thresholds it is labelled as normal; otherwise, it is labelled as aberrant. Thresholds are defined for each individual. To estimate the likelihood of infection, Bayesian statistics were used against the accumulated evidence of aberrant heart rate patterns over time and makes an assessment of the likelihood of infection, based on the accumulated evidence.

If subjects do become unwell during the 26-week period, they are requested to continue, as much as possible, the data collection and wear the smartwatch during their regular schedule and annotate any symptoms, medical diagnosis, and treatment in the daily wellness updates.

Data to be collected from the study volunteers include: 1) time periods when the smartwatch was collecting vital-sign data, 2) heart-rate data from the smartwatch, 3) temperature data from the smartwatch, 4) 3-axis accelerometer data from the smartwatch, 5) any raw sensor data collected by the smartwatch, 6) any self-reported wellness information (e.g., diagnosed illness, fever, or other health concerns), and 7) the algorithm outputs: aberrant heart-rate values and predicted likelihood of infection. Volunteers will be required to follow a consistent daily schedule in terms of when they wear and charge their smartwatch. Device usage will be assessed monthly during the monthly data download visit.

To determine the sample size for the proposed study, the minimum number of volunteers (N) required to achieve statistical significance for the sensitivity and specificity of the model in the prediction of abnormal cases (which may be caused by COVID-19, influence, or common cold) among the volunteers in a period of six months will be sought. The plan is to predict a volunteer's status as abnormal or normal once a week. This gives a total of 24N predictions in six months (26 weeks minus 2 weeks used for establishing the baseline HR values). For a given sensitivity Sₑ, specificity Sₚ, and a number of true abnormal cases K, we will have M positive predictions, where

$$M = {S}_{e} K + [ 1 - {S}_{p} ] [ ( 24 N ) - K ] .$$

Among the M positive predictions, the number of true abnormal cases is x=SₑX. The probability of obtaining true abnormal cases larger than x by randomly choosing M out of 24N cases can be calculated by the cumulative density function of a hypergeometric distribution:

$$p ( X \geq x ) = 1 - {\sum }_{i = 0}^{x - 1} \frac{( \begin{matrix}K \\ i\end{matrix} ) ( \begin{matrix}24 N - K \\ M - i\end{matrix} )}{( \begin{matrix}24 N \\ M\end{matrix} )} .$$

Here, p is also the significance value (i.e., the p value) for a given sensitivity and specificity. Thus, we identified the smallest number of volunteers to guarantee a p value <0.01.

In a previous study, vital-sign data was collected using the Samsung Gear S3 for 10 subjects infected with malaria. Preliminary analysis of the vital-sign data suggests that the infection can be detected at, or prior to, the onset of malaria symptoms in 75% of the cases. Therefore, in this study, the assumption is that our method's sensitivity and specificity are >70%. Using these values, the number of abnormal cases (K) can be estimate, assuming an incidence rate of 2.46% for COVID-19 in a period of six months [based on the fraction of COVID-19 cases observed in Montgomery County, Md., U.S., through roughly the summer of 2020], 8% for influenza during a flu season [Tokars et al., 2018], and two common colds per person per six-month period [Worrall, 2011].

Based on the above assumptions, it was determined N=20 volunteers. This is based on a total number of true abnormal cases K=12, with zero COVID-19 cases, two with flu cases, and ten common colds (which is about 25% of the expected number of common cold cases). Using the equation above with N=20, K=12, and M=141 (computed per the first equation), a p value <0.01 was obtained, for a sensitivity and specificity of 70% or greater. In order to be able to include COVID-19 cases and to further assure a large enough sample size, the plan is to recruit 40 volunteers for this study.

For data collection, the total number of hours, per day, for each subject will be calculated for each day in the study on a monthly basis after each data download. A compliance rate will be determined for each subject based on the percentage of days in the study where the subject collected at least 12 hours of vital-signs and activity data. Additionally, usage patterns will be assessed, such as the time windows during which the vital-signs and activity data were collected.

The output of the model will not be visible to the volunteer but the model will be running as vital-sign data are transferred to the smartphone. After each monthly download, the vital-sign data and wellness reports will be processed to evaluate the performance of the model.

H. Shigella Study

A study has been proposed to test the model as part of a Shigella study with the U.S. Navy tentatively planned for the first half of 2022. This study will be a human controlled study similar to the malaria study discussed above.

I. Observations

It is believed that infectious diseases that can be detected by at least one embodiment described in this disclosure, include but are not limited to, Virus Protease Associated Disease Picornaviridae Enterovirus 3C (CSL), 2A(CSL) Meningitis, gastro-intestinal infections Coxsackievirus 3C Common cold Echovirus 3C Summer flu, hand-foot-and mouth Poliovirus 3C Poliomyelitis Rhinovirus 3C (CSL), 2A(CSL) Common cold, asthma exacerbation in allergies Aphthovirus 3C (CSL), L (Cys) Foot and mouth disease Cardiovirus 3C (CSL) Encephalitis, heart disease (mainly murine, affects other mammals) Hepatovirus 3C (CSL) Hepatitis A (chronic jaundice) Togoviridae Alphaviruses Cys and Ser Equine encephalitis Poxyviruses Ser Smallpox Rubiviruses Cys Rubella (German measles) Paramyxoviridae Parainfluenza Ser Respiratory infection RSV Ser Infant bronchiolitis, viral pneumonia Coronaviridae Cys, 3C-like CSL, Ser Infant bronchiolitis, viral pneumonia SARS Cys 3C like, SAR CoV 3C like, SARS-CoV-2, SARS CoV 2, PL1 PL2 Acute respiratory syndrome Arterivirus Cys Pig disease Flaviviridae Flavivirus NS3 (Ser, unique), NS2B (Ser, unique) Yellow-fever NS3 (Ser, unique) Yellow-fever virus HepC virus NS3 (Ser, unique) Hepatitis C Pestivirus Cys, Ser Pigs, cattle and sheep disease Adenoviridae Acute upper respiratory, eye and intestinal tract, infant death Herpesviridae Herpesviridae Herpes (systemic and topical) Retroviridae HIV Asp AIDS Caliciviridae 3C-like (CSL) Rabbit hemorrhagic disease Potyviridae Potyvirus NIa (3C-like CSL), malaria and combinations thereof.

While specific aspects of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

Although the methods discussed in this disclosure with reference to particular flowcharts, it should be understood that the order of the steps shown to be varied from the order illustrated in other embodiments, that steps discussed as being separate can be combined, and that not all steps discussed are necessarily required in all embodiments. Additionally, in at least one embodiment where the implementation uses a processor, the processor executes code for the steps as such is an example of means for performing the discussed function.

While specific embodiments of the invention were illustrated and discussed regarding the application of the principles of the invention, it will be understood that the invention may be embodied otherwise without departing from such principles.

In the claims that follow this specification, it should be understood that the invention includes placing the claims into nested (or multiple levels) of multiple dependency form where no conflict exists between dependent claims.

### VI. INDUSTRIAL APPLICABILITY

The above-described devices, systems, and methods have use in assisting individuals and/or companies determining when an individual may have a pre-symptomatic adverse condition such as an infection to allow for an early intervention before the individual is exhibiting symptoms, for example by providing medical care, contact tracing, and/or isolating.

### VII. REFERENCES DISCUSSED IN THE STUDY DISCUSSION SECTION

1. Spring, M., M. Polhemus, and C. Ockenhouse, Controlled human malaria infection. J Infect Dis, 2014. 209 Suppl 2: p. S40-5.
2. Bartoloni, A. and L. Zammarchi, Clinical aspects of uncomplicated and severe malaria. Mediterr J Hematol Infect Dis, 2012. 4(1): p. e2012026.
3. Chulay, J. D., et al., Malaria transmitted to humans by mosquitoes infected from cultured Plasmodium falciparum. Am J Trop Med Hyg, 1986. 35(1): p. 66-8.
4. Epstein, J. E., et al., Safety and clinical outcome of experimental challenge of human volunteers with Plasmodium falciparum-infected mosquitoes: an update. J Infect Dis, 2007. 196(1): p. 145-54.
5. Hermsen, C. C., et al., Testing vaccines in human experimental malaria: statistical analysis of parasitemia measured by a quantitative real-time polymerase chain reaction. Am J Trop Med Hyg, 2004. 71(2): p. 196-201.
6. Gani, A., et al., Universal glucose models for predicting subcutaneous glucose concentration in humans. IEEE Trans Inf Technol Biomed, 2010. 14(1): p. 157-65.
7. Laxminarayan, S., et al., Human core temperature prediction for heat-injury prevention. IEEE J Biomed Health Inform, 2015. 19(3): p. 883-91.
8. Reifman, J., et al., 2B-Alert Web: an open-access tool for predicting the effects of sleep/wake schedules and caffeine consumption on neurobehavioral performance. Sleep, 2016. 39(12): p. 2157-2159.
9. Reisner, A. T., et al., Muscle oxygen saturation improves diagnostic association between initial vital-signs and major hemorrhage: a prospective observational study. Acad Emerg Med, 2016. 23(3): p. 353-7.
10. Yu, C., et al., QuartetS: a fast and accurate algorithm for large-scale orthology detection. Nucleic Acids Res, 2011. 39(13): p. e88.
11. Chen, L., et al., Are standard diagnostic test characteristics sufficient for the assessment of continual subject monitoring? Med Decis Making, 2013. 33(2): p. 225-34.
12. Marple, S. L., Digital Spectral Analysis With Applications. 1987, Englewood Cliffs: Prentice-Hall.
13. Neumaier, A. and T. Schneider, Estimation of parameters and eigenmodes of multivariate autoregressive models. ACM Transactions on Mathematical Software, 2001. 2(1): p. 27-57.
14. Wald, T., Sequential tests of statistical hypotheses. Ann Math Stat, 1947. 162(2): p. 117.
15. Dutta, B., A. Wallqvist, and J. Reifman, PathNet: a tool for pathway analysis using topological information. Source Code Biol Med, 2012. 7(1): p. 10.
16. Kumar, K., et al., AGeS: a software system for microbial genome sequence annotation. PLOS ONE, 2011. 6(3): p. e17469.
17. Vijaya Satya, R., et al., A high-throughput pipeline for the design of real-time PCR signatures. BMC Bioinformatics, 2010. 11: p. 340-49.
18. Vijaya Satya, R., N. Zavaljevski, and J. Reifman, SNIT: SNP identification for strain typing. Source Code Biol Med, 2011. 6: p. 14-18.
19. Yu, C., et al., The development of PIPA: an integrated and automated pipeline for genome-wide protein function annotation. BMC Bioinformatics, 2008. 9: p. 52-62.
20. Yu, C., et al., Genome-wide enzyme annotation with precision control: catalytic families (CatFam) databases. Proteins, 2009. 74(2): p. 449-60.
21. Ramakrishnan, S., et al., A unified model of performance for predicting the effects of sleep and caffeine. Sleep, 2016. 39(10): p. 1827-41.
22. Liu, J., et al., Automated analysis of vital-signs to identify subjectssubjects with substantial bleeding before hospital arrival: a feasibility study. Shock, 2015. 43(5): p. 429-436.
23. S. Laxminarayan et al. “Individualized estimation of human core body temperature using noninvasive measurements,” J Appl Physiol (1985), vol. 124, no. 6, pp. 1387-1402, Jun. 1, 2018.

What is claimed is:

1. A method for determining a probability of an infection for an individual using a baseline mean, a baseline standard deviation and a threshold for a heart rate of the individual being monitored, the method comprising:
collecting heart rate data and activity information for the individual for a monitoring time window using at least one sensor adapted to be worn by the individual;
converting the collected activity information to an activity level for the monitoring time window;
selecting a time window and activity bin based on the activity level and the monitoring time window for the collected heart rate data from at least one sensor;
calculating a heart rate score based on the heart rate data for the monitoring time window and the baseline mean and the baseline standard deviation for the selected bin;
classifying the heart rate score as normal or aberrant based on how the heart rate score compares with the threshold;
calculating a probability using the classification as normal/aberrant, aberrant heart rate probabilities for a healthy person and an infected person, and after the first probability calculation, the previous probability;
providing the probability to the individual and/or another individual to alert at least one of the individuals of the likelihood of the infection when the probability exceeds a probability threshold, the individual and/or the other individual acts in response to the provided probability; and
repeating the above steps for at least one more monitoring time window.

2. The method according to claim 1, wherein the individual acts by obtaining a medical diagnostic test, obtaining a medical examination, and/or isolating, and/or the other individual acts by adjusting a work schedule for the individual and/or coworkers of the individual.

3. The method according to claim 1, further comprising when the monitoring time window includes activity for the individual from multiple activity levels, subdividing the monitoring time window based on those activity levels and calculating the heart rate score for each subdivision to determine whether each subdivision is aberrant or normal, and
wherein the total number of aberrant or normal determinations for one monitoring time window is between 1 and the number of activity levels.

4. The method according to claim 1, wherein the probability threshold is set at a level appropriate for an infection frequency in an area in which the individual is present such that the probability threshold is the lower of 50% or 100% minus a current infection rate.

5. The method according to claim 1, wherein the heart rate score S is
$$S ( {t}_{i} , {a}_{j} ) = \frac{H R ( {t}_{i} , {a}_{j} ) - m ( {t}_{i} , {a}_{j} )}{S D ( {t}_{i} , {a}_{j} )}$$
where tᵢ is the monitoring time window i in a day, aⱼ is the activity level j, HR is a mean of the heart rates based on heart rate data for that time window and activity level, and for the selected bin, m is the baseline mean and SD is the baseline standard deviation.

6. The method according to claim 1, wherein the probability is
$${p}_{t} = \frac{1}{1 + {( \frac{{p}_{h}}{{p}_{i}} )}^{n} {( \frac{1 - {p}_{h}}{1 - {p}_{i}} )}^{k} ( \frac{1}{{p}_{t - 1}} - 1 )}$$
where pₕ is a probability to observe an aberrant heart rate in a healthy subject, pᵢ is a probability to observe an aberrant heart rate in an infected subject, t is the monitoring time window, and n and k, respectively, are the number of aberrant and normal heart rates observed in the monitoring time window.

7. The method according to claim 6, wherein the monitoring time window has a length equal to 15 minutes, 30 minutes, 45 minutes, 60 minutes, 90 minutes, or 120 minutes.

8. The method according to claim 6, wherein the prior probability is multiplied by a multiplier smaller than one, resulting in past aberrant scores impacting the current probability less over time.

9. The method according to claim 1, wherein when the baseline mean and the baseline standard deviation are not available for the selected time window and activity bin, using at least one of a prior time window and activity bin and a later time window and activity bin.

10. A method for establishing baselines for an individual using a heart rate of the individual, the method comprising:
collecting baseline heart rate data and activity information for the individual over multiple days using at least one heart rate sensor and at least one activity monitor;
separating the heart rate data into bins for baseline time windows based on a time of day and an activity level derived from the activity information; and
determining a baseline mean and a baseline standard deviation for the heart rates for each bin and at least one threshold based on all of the baseline heart rate data and activity information.

11. The method according to claim 10, wherein
the at least one activity monitor includes a 3-axis accelerometer, and
there are at least three activity levels for each time window, each bin having a preset range of metabolic equivalents (METs) or activity as computed from the activity information from the 3-axis accelerometer.

12. The method according to claim 10, wherein for each time window with baseline heart rate data and activity information, calculating a heart rate score to determine the at least one threshold.

13. The method according to claim 12, wherein the heart rate score for one time window is based on the heart rate for that time window, the mean heart rate and the standard deviation for the bin corresponding to the time window and the activity level.

14. The method according to claim 12, wherein the heart rate score S is
$$S ( {t}_{i} , {a}_{j} ) = \frac{H R ( {t}_{i} , {a}_{j} ) - m ( {t}_{i} , {a}_{j} )}{S D ( {t}_{i} , {a}_{j} )}$$
where tᵢ is the baseline time window i in a day, aⱼ is the activity level j, HR is a mean of the heart rates in the heart rate data for that time window, and for that bin, m is the baseline mean and SD is the baseline standard deviation.

15. The method according to claim 10, further comprising
creating a distribution of the heart rate scores,
selecting a lower heart rate score and a higher heart rate score from the heart rate scores that represent a lower threshold percentage or number and a higher threshold percentage or number, respectively,
calculating a lower threshold using the lower heart rate score, and
calculating a higher threshold using the higher heart rate score.

16. The method according to claim 10, further comprising after the baseline mean, the baseline standard deviation, and the threshold are determined, ongoing monitoring of the individual by
collecting ongoing heart rate data and activity information for a monitoring time window for the individual using the at least one heart rate sensor and the at least one activity monitor,
selecting a monitoring time window and activity bin based on activity information for the monitoring time window,
calculating a heart rate score based on the heart rate data for the monitoring time window and the baseline mean and the baseline standard deviation for the time window and activity bin corresponding to the monitoring bin,
classifying the heart rate score as normal or aberrant based on how the heart rate score compares with the at least one threshold,
calculating a probability using the classification as normal/aberrant, aberrant heart rate probabilities for a healthy person and an infected person, and after the first probability calculation, the previous probability,
providing the probability to the individual and/or another individual to alert at least one of the individuals of the likelihood of the infection when the probability exceeds a probability threshold, the individual and/or the other individual acts in response to the provided probability to address any infection probability of the individual, and
repeating the ongoing monitoring steps

17. The method according to claim 16, further comprising:
alerting the individual when the probability exceeds a probability threshold to allow the individual to seek medical assistance or take other action; and
when the monitoring time window includes activity for the individual from multiple activity levels, subdividing the monitoring time window based on the corresponding activity levels and calculating the heart rate score for each subdivision to determine whether each subdivision is aberrant or normal, and
wherein the total number of aberrant or normal readings for one monitoring time window is between 1 and the number of subdivisions.

18. The method according to claim 16, wherein at least one non-heart rate vital-sign for the individual is concurrently monitored with the heart rate,
the method further comprising:
establishing a baseline mean, a baseline standard deviation, and a threshold range for the non-heart rate vital-sign,
monitoring the non-heart rate vital-sign,
calculating a non-heart rate vital-sign score,
calculating a second probability for the non-heart rate vital-sign score and
combining the first and second probabilities together to be an overall probability by averaging together or combining the probabilities based on predetermined weights, wherein the overall probability is provided.

19. A system for informing an individual of a probability of an infection based on heart rate data for the individual, the system comprising:
at least one wearable device having at least one heart rate monitor and/or an activity module;
a computing device in wireless communication with said at least one wearable device, said computing device having a display and a processor configured to
receive baseline heart rate data and activity information for the individual over multiple days from said at least one wearable device;
separate the heart rate data into time window and activity bins based on a time of day for the heart rate data and an activity level derived from the activity information;
determine a baseline mean and a baseline standard deviation for the heart rate data for at least each bin with baseline heart rate data and at least one threshold based on all of the baseline heart rate data and activity information; and
after the baseline mean, the baseline standard deviation, and the threshold are determined,
receive monitoring heart rate data and activity information for a monitoring time window for the individual from said at least one wearable device,
select a monitoring bin based on received activity information and a time of day of the monitoring time window,
calculate a heart rate score based on the heart rate data for the monitoring time window and the baseline mean and the baseline standard deviation for the baseline bin that corresponds to the selected monitoring bin,
classify the heart rate score as normal or aberrant based on how the heart rate score compares with the threshold,
calculate a probability using the classification as normal/aberrant, an aberrant heart rate probability for a healthy person, an aberrant heart rate probability for an infected person, and after the first probability calculation, the previous probability, and
display the probability on said display.

20. The system according to claim 19, wherein
the activity module includes a 3-axis accelerometer and generates a metabolic equivalents (METs) value and/or accelerometer data to facilitate separation of the heart rate data into the activity bins;
the baseline time window and/or the monitoring time window has a length equal to 15 minutes, 30 minutes, 45 minutes, 60 minutes, 90 minutes, or 120 minutes;
the heart rate score S is
$$S ( {t}_{i} , {a}_{j} ) = \frac{H R ( {t}_{i} , {a}_{j} ) - m ( {t}_{i} , {a}_{j} )}{S D ( {t}_{i} , {a}_{j} )}$$
where tᵢ is the time window i in a day, aⱼ is the activity level j, HR is a mean of the heart rate measurements contained in the heart rate data for that time window, and for the selected monitoring bin, m is the baseline mean and SD is the baseline standard deviation;
the probability is
$${p}_{t} = \frac{1}{1 + {( \frac{{p}_{h}}{{p}_{i}} )}^{n} {( \frac{1 - {p}_{h}}{1 - {p}_{i}} )}^{k} ( \frac{1}{{p}_{t - 1}} - 1 )}$$
where pₕ is a probability to observe an aberrant heart rate in a healthy subject, pᵢ is a probability to observe an aberrant heart rate in an infected subject, t is the monitoring time window, and n and k, respectively, are the number of aberrant and normal heart rates observed in the monitoring time window; and
the prior probability is multiplied by a multiplier resulting in past aberrant scores impacting the current probability less over time.
